(19) United States
(12) Patent Application Publication
(10) Pub. No.: US 2022/0098584 A1
(43) Pub. Date: Mar. 31, 2022

(54) ANTISENSE OLIGONUCLEOTIDES FOR THE TREATMENT OF LEBER`S CONGENITAL AMAUROSIS

(71) Applicant: ProQR Therapeutics II B.V., Leiden (NL)

(72) Inventors: Jim Swildens, Leiden (NL); Jeroen van de Giessen, Leiden (NL); Kalyana Chakravarthi Dulla, Leiden (NL)

(21) Appl. No.: 17/423,620

(22) PCT Filed: Jan. 27, 2020
(86) PCT No.: PCT/EP2020/051942

§ 371 Date: Jul. 16, 2021

(30) Foreign Application Priority Data
Jan. 28, 2019 (EP) 19153889.1
Apr. 16, 2019 (EP) 19169523.8

(51) Int. Cl. C12N 15/113; A61P 27/02; A61K 31/7125

(52) U.S. Cl. CPC C12N 15/113; A61P 27/02; A61K 31/7125; C12N 2320/32; C12N 2320/33; C12N 2310/322; C12N 2310/11

(57) ABSTRACT

The invention relates to antisense oligonucleotides (AON) capable of inducing the skip of exon 36 from human CEP290 pre-mRNA. The c.4723A>T mutation in the human CEP290 gene is the cause of Leber's Congenital Amaurosis type 10 (LCA10) in patients carrying this mutation. The AONs of the present invention can be used in the treatment of LCA10 caused by mutations in exon 36, such as the c.4723A>T mutation. The invention relates to AONs, pharmaceutical formulations comprising such AONs, and viral vectors expressing such AONs, that may be used in the treatment of LCA10.

### FIELD OF THE INVENTION

The invention relates to the field of medicine. More in particular, it relates to the field of antisense oligonucleotides that are used in the treatment of Leber's Congenital Amaurosis type 10 (LCA10). More specifically, the invention relates to antisense oligonucleotides that induce skipping of exon 36 from human CEP290 (pre-) mRNA.

### BACKGROUND OF THE INVENTION

Leber's Congenital Amaurosis (LCA) is the most common form of congenital childhood blindness with an estimated prevalence of approximately 1 in 50,000 new-borns, worldwide. It is accompanied by retinal dystrophy. The diagnosis of LCA is usually made in the first months of life in an infant presenting with congenital nystagmus, sluggish photomotor reflex, oculo-digital signs of Franceschetti, inability to follow light or objects and normal fundus. Genetically, LCA is a heterogeneous disease, with eighteen genes identified to date in which mutations are causative for LCA. The most frequently mutated LCA gene is CEP290, a gene located on the Q arm of chromosome 12, coding for Centrosomal Protein 290 (CEP290), which has an important role in centrosome and cilia development. CEP290 is vital in the formation of the primary cilium, a small antenna-like projection of the cell membrane that plays an important role in photoreceptors at the back of the retina (which detect light and colour) and in the kidney, brain, and many other organs of the body. Knocking down levels of the CEP290 gene transcript resulted in dramatic suppression of ciliogenesis in retinal pigment epithelial cells in culture, proving just how important CEP290 is to cilia formation. The disease caused by CEP290 mutations is referred to as LCA type 10, or LCA10. Mutations in the CEP290 gene are responsible for about 15% of all LCA cases. The most frequently occurring CEP290 mutation, associated with retinal dystrophy, especially in the Western world, is a change in intron 26 of the CEP290 gene: c.2991+1655A>G, which creates a cryptic splice donor site in intron 26 that results in the inclusion of a pseudoexon of 128 bp in the mutant CEP290 mRNA. This inclusion of the aberrant exon introduces a premature stop codon (p.C998X). In patients with this mutation, the wild type transcript that lacks the aberrant exon is still produced, explaining the hypomorphic nature of this mutation. Antisense oligonucleotides (AONs) that target the mutated CEP290 pre-mRNA and that prevent the inclusion of the 128 bp aberrant exon have been disclosed in the art (U.S. Pat. Nos. 9,771,580; 10,167,470; 9,012,425; 9,487,782; 9,777,272; WO 2016/034680; WO 2016/135334). Clinical trials have shown the efficacy in using an AON for the treatment of LCA10 in humans (QR-110; sepofarsen; WO 2016/135334).

To date, over a hundred CEP290 mutations have been identified leading to a spectrum of phenotypes ranging from isolated early-onset retinal dystrophy and LCA, to more severe syndromes such as Senior Løken syndrome, Joubert syndrome or Meckel-Gruber syndrome. The c.4723A>T mutation located in exon 36 of the human CEP290 gene also causes LCA10. This mutation (also referred to as p.(Lys1575X)) introduces a premature stop codon. Initial estimates indicate that the number of patients carrying this mutation (either in homozygosity or compound heterozygosity with c.2991+1655A>G or another mutation) may range between 200-400 patients in the Western world. An incidence of 28% relative to the c.2991+1655A>G mutation has been reported (Perrault et al. 2007. Spectrum of NPHP6/CEP290 mutations in Leber Congenital Amaurosis and delineation of the associated phenotype. Hum. Mutat. 28:416-425). Exon 36, consisting of 108 bp, in the human CEP290 gene is in-frame with exon 35 and exon 37, which means that skipping exon 36 would yield an in-frame transcript. In fact, it has been shown that in in healthy individuals exon 36 is sometimes skipped from the CEP290 pre-mRNA, which indicates that a splice variant of CEP290 in which this part of the protein is absent exists in nature and is likely (partly) functional (Roosing et al. 2017. A rare form of retinal dystrophy caused by hypomorphic nonsense mutations in CEP290. Genes 8:208). The CEP290 protein lacking the translated part of exon 36 maintains basal function and results in significantly less severe non-syndromic manifestation. WO 2015/004133 discloses two antisense oligonucleotides (20-mer 2′-O-methyl modified m36ESE and 24-mer 2′-O-methyl modified m36D) that were transfected into mouse NIH-3T3 fibroblast cells to target the wild type mouse Cep290 pre-mRNA. It should be noted that in literature it is often noted that exon 36 in human CEP290 is the equivalent of exon 35 in mouse Cep290, and in a follow-up publication (Gerard et al. 2015. Intravitreal injection of splice-switching oligonucleotides to manipulate splicing in retinal cells. Nucleic Acids 4:e250) the same oligonucleotides were respectively referred to as m35ESE and m35D. In that publication m35ESE was also injected into wild type C57BL/6J mouse eyes and exon skipping of exon 35 was detected in the mouse retina, whereas m35D was not tested. Despite these efforts there remains a need for efficient and improved medicaments that can target the human mutant CEP290 pre-mRNA and that are applicable in treating LCA10 in human subjects that suffer from mutations in one or both exons 36 of their CEP290 alleles. Currently there is no cure or treatment available for patients carrying the c.4723A>T mutation in that exon.

### SUMMARY OF THE INVENTION

The present invention relates to an antisense oligonucleotide (AON) capable of inducing skipping exon 36 from human CEP290 (pre-) mRNA, wherein the AON comprises or consists of a sequence that is substantially complementary to a sequence within exon 36 of the human CEP290 gene. Particularly preferred are AONs that consist of 15, 16, 17, 18, 19 or 20 nucleotides and that are substantially, more preferably 100%, complementary to a consecutive sequence within SEQ ID NO:147. In another aspect, the invention relates to an AON capable of inducing exon 36 skipping, wherein the AON comprises a sequence that is substantially, more preferably 100%, complementary to a sequence within exon 36 of the human CEP290 gene and overlaps with the 5′ or the 3′ intron/exon boundary of exon 36, more preferably with the exon 36/intron 36 boundary at the 3′ end of exon 36 and the 5′ end of intron 36. In one embodiment, the AON of the present invention consists of a sequence selected from the group consisting of: SEQ ID NO:7, 8, 11, 12, 15, 16, 18, 19, 26, 27, 28, 29, 37, 38, 39, 40, 41, 51, 52, 53, 54, 55, 56, 57, 58, 59, 60, 61, 63, 64, 65, 66, 67, 70, 71, 72, 74, 75, 76, 77, 78, and 93 to 146. More preferably, the AON of the present invention consists of a sequence selected from the group consisting of: SEQ ID NO:7, 8, 12, 19, 26, 27, 28, 29, 39, 53, 54, 55, 56, 57, 58, 60, 61, 74, 75, 76, 77, 78, and 93 to 146. Even more preferably, the AON of the present invention consist of a sequence selected from the group consisting of: SEQ ID NO:53, 54, 55, 56, 57, 58, 61, 74, 75, 76, 77, 78, and 93 to 146. Most preferably, the AON of the present invention consists of a sequence selected from the group consisting of: SEQ ID NO:53, 54, 55, 56, 58, 74, 75, 76, 77, 78, 105, 106, 125, 126, 143, 144, 145, and 146. The AON of the present invention preferably comprises at least one non-naturally occurring chemical modification, such as a phosphorothioate linkage, and/or a mono- or di-substitution at the 2′, 3′ and/or 5′ position of the sugar moiety, such as a 2′-OMe modification or a 2′-MOE modification. In a more preferred aspect, the AON according to the present invention carries a 2′-MOE modification in each sugar moiety. In another particularly preferred aspect, the AON according to the present invention carries a 2′-OMe modification in each sugar moiety. In yet another preferred embodiment, the AON of the present invention has a fully phosphorothioated backbone.

The present invention also relates to a pharmaceutical composition comprising an AON according to the invention, and a viral vector expressing an AON according to the invention. In one aspect, the AON, the pharmaceutical composition, or the viral vector according to the invention are for use as a medicament, preferably for the treatment, prevention or delay of a CEP290-related disease or a condition requiring modulating splicing of human CEP290 pre-mRNA, such as Leber's Congenital Amaurosis type 10 (LCA10).

The invention also relates to a use of an AON, a pharmaceutical composition, or a viral vector according to the invention for the preparation of a medicament for the treatment, prevention or delay of a CEP290-related disease or condition requiring modulating splicing of CEP290 pre-mRNA, such as LCA10. The invention in another aspect, relates to a method for modulating splicing of CEP290 pre-mRNA in a cell, or to a method for the treatment of a CEP290-related disease or condition requiring modulating splicing of CEP290 pre-mRNA of an individual in need thereof, using an AON, a pharmaceutical composition, or a viral vector according to claim the invention.

### BRIEF DESCRIPTION OF THE DRAWINGS

FIG. 1 shows the CEP290 exon 36 (bold, upper case) sequence and a part of its surrounding intron DNA sequences (bold, lower case), from 5′ to 3′. Displayed is the mutated sequence (SEQ ID NO:1), with the c.4723A>T mutation indicated by an asterisk (*). The target region that was identified herein to be of importance (spanning a region comprising a 3′ part of exon 36 and a 5′ part of downstream intron 36) is underlined (SEQ ID NO:147). The bold DNA sequences in FIGS. 1A, B, C and D are the same. Below the CEP290 sequence are given the sequences of the complementary antisense oligonucleotides (AONs) disclosed herein, each here shown from 3′ to 5′ (left to right). The skilled person knows that the AONs target the transcribed (pre-) mRNA, even though the DNA sequence is provided here. AONs QRX136.30, -31, -32, -33, and -34 target a region including the c.4723A>T mutation with a complementary adenosine (A) at the position of the mutation (i.e., opposite a T (or a U in RNA) in the c.4723A>T mutant situation). QRX136.30a, -33a, -34a, and -54a carry a complementary uridine (U) at the position of the mutation (i.e. opposite an A in the wild type situation), to test their exon 36-skipping capability in wild type cells. All AONs given here may come in a variety of modified versions. However, in general, as disclosed herein the AONs were tested either in a fully 2′-OMe modified or in a fully 2′-MOE modified version with a phosphorothioated backbone. The “a” in the name of the AON only indicates that the AON carries 2′-MOE modifications instead of 2′-OMe modifications (no “a”), but clearly the sequence would not change when the modification at the 2′ position of the sugar moiety would change, and the name solely serves as a reference. Given are also the sequences of AONs known from the art: m35ESE and m35ESEa are complementary to exon 35 of the mouse Cep290 gene, which is the equivalent of human exon 36 in human CEP290. The non-complementary nucleotides in the mouse Cep290-targeting AONs are underlined. Their equivalents for human CEP290 targeting are referred to as h35ESE and h35ESEa, respectively. These AONs target exon 36 in human CEP290. The same holds true for m35D and m35 Da that overlap with the 3′ end of mouse exon 35 in Cep290 and its downstream intron sequence. Their equivalents for human CEP290 targeting are referred to as h35D and h35 Da, respectively. These AONs also target exon 36 in human CEP290. H36D and H36 Da are AONs published after the priority date of the present invention (Barny et al. 2019. AON-mediated exon skipping to bypass protein truncation in retinal dystrophies due to the recurrent CEP290 c.4723A>T mutation. Fact or fiction? Genes 10:368).

FIG. 2 shows the percentages of exon 36 skip from human CEP290 mRNA using twenty-six 2′-OMe modified antisense oligonucleotides (QRX136.27 to QRX136.52 as depicted) after a transfection in wild type human fibroblasts and subsequently assayed with ddPCR.

FIG. 3 shows the percentages of exon 36 skip from human CEP290 mRNA using ten additional 2′-MOE modified antisense oligonucleotides (QRX136.29a, -53a, -30a, -33a, -54a, -34a, -48a, -49a, -50a, and -51a as depicted together with a negative control AON) after transfection in wild type human fibroblasts and subsequently assayed with ddPCR.

FIG. 4 shows the percentages of exon 36 skip from human CEP290 mRNA using additional (shorter) 2′-MOE modified AONs (QRX136.55a to QRX136.65a; open bars) in comparison to 22-mer 2′-MOE modified AONs tested before (QRX136.34a, QRX136.48a and QRX136.50a; black bars) after transfection in wild type human WERI-Rb-1 cells and subsequently assayed with ddPCR.

FIG. 5 shows the percentages of exon 36 skip from human CEP290 mRNA using the same AONs as shown in the previous figure, after gymnotic uptake in wild type human WERI-Rb-1 cells, and subsequently assayed with ddPCR, showing that QRX136.58a and QRX136.59a outperformed the other AONs. Black bars represent AONs tested earlier; open bars represent AONs newly tested.

FIG. 6 shows the percentages of exon 36 skip from human CEP290 mRNA using the AONs shown as depicted in comparison to several published AONs (10 μM each), after gymnotic uptake in wild type human WERI-Rb-1 cells and subsequently assayed with ddPCR. The known mouse-specific m35ESE oligonucleotide was prepared in a fully 2′-MOE modified version (m35ESEa), while based on the sequence of m35ESE also a human-specific version was generated in a 2′-OMe version (h35ESE) and in a 2-MOE version (h35ESEa). The known mouse-specific m35D oligonucleotide was prepared in a fully 2′-MOE modified version (m35 Da), while based on the sequence of m35D also a human-specific version was generated in a 2′-OMe version (h35D) and in a 2-MOE version (h35 Da). H36D and H36 Da are the human-specific oligonucleotides disclosed by Barney et al. (2019) and refer to a fully 2′-OMe and a fully 2′-MOE modified version. On the right side of the graph the results are depicted from an experiment in which several AONs as depicted with used in combination with a total concentration of 10 μM oligonucleotide. These results show that several new oligonucleotides outperformed the m35ESEa, m35 Da and H36 Da oligonucleotides, while especially the AONs that targeted the human CEP290 (pre-) mRNA in a region that terminates exactly at the exon 36 3′ splice donor site (QRX136.58a, QRX136.59a and QRX136.78a; see FIG. 1) with QRX136.59a performing best, giving almost a 3-fold higher skip percentage than the best performing AON from the art, after gymnotic uptake that represents natural cell entry better than transfection procedures.

FIG. 7 shows the percentages of exon 36 skip in human wild type retinal organoids (optic cups) after incubation with three AONs (QRX136.34a, QRX136.48a and QRX136.61a, respectively) in two different concentration ranges: ‘3 μM’ indicates the treatment with 1.5 μM for 10 days, then followed by 3 μM up to 28 days, ‘9 μM’ indicates the treatment with 6 μM for 10 days, then followed by 9 μM up to 28 days, ‘6 μM’ indicates the continuous treatment of the control AON in a concentration of 6 μM; present in the culture medium). Skip percentages were calculated to be between 28% and 36%. The control, non-targeting AON did not give any skip.

### DETAILED DESCRIPTION OF THE INVENTION

The present invention relates to antisense oligonucleotides (AONs) and the use thereof in the treatment of Leber's Congenital Amaurosis type 10 (LCA10). More in particular it relates to AONs that induce skipping of exon 36 from the human CEP290 (pre-) mRNA. One particular mutation causing LCA10 is the c.4723A>T mutation that generates a premature stop codon in the CEP290 transcript, and which is located in exon 36. The inventors of the present invention reasoned that skipping exon 36 by using AONs would yield a (partly) functional CEP290 protein alleviating the disease. By no means could it be predicted whether any AONs would be identifiable and be capable of tricking the spliceosome and whether these AONs would yield an exon 36 skip from human CEP290 (pre-) mRNA. But, surprisingly, the inventors did identify such AONs that appeared sufficiently effective in obtaining substantial exon 36 skipping. Hence, the inventors of the present invention aimed to identify AONs for exon 36 skipping, succeeded and thereby provide a tool that could be used as a medicament in the treatment of LCA10 in patients that carry one or multiple mutations in exon 36 of one or both of their CEP290 alleles.

In a first aspect, the invention relates to an oligonucleotide capable of reducing the inclusion of exon 36 in the human CEP290 mRNA, wherein the oligonucleotide is complementary to and capable of binding under physiological conditions to the human CEP290 pre-mRNA in a region of exon 36 and/or its surrounding sequences that affect the inclusion of exon 36 in the human CEP290 mRNA. Preferably, the oligonucleotide is complementary to and binds under physiological conditions to a sequence in exon 36 of the human CEP290 pre-mRNA, and/or to a sequence that it includes the boundary with the intron sequence at the 5′ or at the 3′ end of exon 36.

In one aspect, the present invention relates to an AON capable of inducing skipping exon 36 in human CEP290 pre-mRNA, wherein the AON is substantially complementary to a sequence within the human exon 36 sequence. In another aspect, the present invention relates to an AON capable of inducing skipping exon 36 in human CEP290 pre-mRNA, wherein the AON is complementary to a 5′ part of the exon 36 sequence and a 3′ part of the preceding intron (herein referred to as intron 35), and therefore overlapping with the intron 35/exon 36 boundary. In yet another embodiment, the present invention relates to an AON capable of inducing skipping exon 36 in human CEP290 (pre-) mRNA, wherein the AON is complementary to a 3′ part of the exon 36 sequence and a 5′ part of the downstream intron (herein referred to as intron 36), and therefore overlapping with the exon 36/intron 36 boundary. In one embodiment, the AON of the present invention consists of a sequence selected from the group consisting of: SEQ ID NO:7, 8, 11, 12, 15, 16, 18, 19, 26, 27, 28, 29, 37, 38, 39, 40, 41, 51, 52, 53, 54, 55, 56, 57, 58, 59, 60, 61, 63, 64, 65, 66, 67, 70, 71, 72, 74, 75, 76, 77, 78, and 93 to 146. More preferably, the AON of the present invention consists of a sequence selected from the group consisting of: SEQ ID NO:7, 8, 12, 19, 26, 27, 28, 29, 39, 53, 54, 55, 56, 57, 58, 60, 61, 74, 75, 76, 77, 78, and 93 to 146. Even more preferably, the AON of the present invention consist of a sequence selected from the group consisting of: SEQ ID NO:53, 54, 55, 56, 57, 58, 61, 74, 75, 76, 77, 78, and 93 to 146. Most preferably, the AON of the present invention consists of a sequence selected from the group consisting of: SEQ ID NO:53, 54, 55, 56, 58, 74, 75, 76, 77, 78, 105, 106, 125, 126, 143, 144, 145, and 146. In an even more preferred aspect, the present invention relates to an AON capable of inducing skipping exon 36 in human CEP290 (pre-) mRNA, wherein the AON is complementary to a 3′ part of the exon 36 sequence and the region of complementarity terminates at the ultimate nucleotide of exon 36 (and the AON starts with its 5′ nucleotide being complementary to the most 3′ nucleotide of exon 36) and does not overlap with the downstream intron 36 sequence. Hence, the most preferred AONs according to the present invention are those that consist of the sequence of SEQ ID NO:53, 54, 55, 74, 105, and 106 (20-mer QRX136.57a, 18-mer QRX136.58a, 16-mer QRX136.59a, 17-mer QRX136.78a, 19-mer QRX136.113a, and 15-mer QRX136.114a, respectively).

In one aspect, the AON of the present invention comprises a sequence that is complementary to a sequence selected from the group consisting of: SEQ ID NO:42, SEQ ID NO:44, SEQ ID NO:48, and SEQ ID NO:147. SEQ ID NO:42 (Box 1) represents the target sequence shared by QRX136.29 (and QRX136.29a), QRX136.30 (and QRX136.30a) and QRX136.53a. SEQ ID NO:44 (Box 2 mutant) represents the target sequence shared by QRX136.33 and QRX136.34, in which the most 5′ position (T) is the c.4723A>T mutation. In comparison and to enable proper exon skipping due to targeting that region in the experiments using wild type cells as exemplified herein, SEQ ID NO:46 represents the Box 2 wild type target sequence shared by QRX136.33a, QRX136.34a and QRX136.54a. SEQ ID NO:48 (Box 3) represents the target sequence shared by QRX136.48, QRX136.49 and QRX136.50. SEQ ID NO:147 represents the target sequence that yielded the most efficient exon 36 skipping oligonucleotides, as shown in the examples provided herein. Using publicly available tools to find potential Exonic Splice Enhancer (ESE) elements, it appeared that Box 1 and Box 2 contain several ESE and ESS elements, whereas Box 3 did not reveal many of these elements (not shown). Based on the findings of the inventors as disclosed herein, together with the tools available to the skilled person, it was anticipated that AONs comprising sequences that target the sequences of these three box regions and SEQ ID NO:147 can influence the splicing of exon 36, and are useful in the treatment of LCA10 in patients harboring LCA10-causing mutations in exon 36. Since other mutations than the c.4723A>T mutation may also be present in exon 36, the present invention also relates to an AON capable of inducing the skipping of exon 36 wherein the AON is complementary to the wild type sequence of Box 2, represented by SEQ ID NO:46. In a preferred aspect, the AON of the present invention therefore comprises or consists of the sequence selected from the group consisting of: SEQ ID NO:43, SEQ ID NO:45, SEQ ID NO:47 and SEQ ID NO:49, which are the sequences complementary to the target sequences of SEQ ID NO:42, 44, 46, and 48, respectively.

The skilled person knows that an oligonucleotide, such as an RNA oligonucleotide, generally consists of repeating monomers. Such a monomer is most often a nucleotide or a nucleotide analogue. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a “scaffold modification”. For severe modifications, the original pentose sugar might be replaced in its entirety by another moiety that similarly connects the base and the phosphate. It is therefore understood that while a pentose sugar is often a scaffold, a scaffold is not necessarily a pentose sugar.

A base, sometimes called a nucleobase, is generally adenine, cytosine, guanine, thymine or uracil, or a derivative thereof. Cytosine, thymine and uracil are pyrimidine bases, and are generally linked to the scaffold through their 1-nitrogen. Adenine and guanine are purine bases and are generally linked to the scaffold through their 9-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked monomers of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate, such a moiety is still referred to as the backbone linkage of the monomer. This is referred to as a “backbone linkage modification”. In general terms, the backbone of an oligonucleotide comprises alternating scaffolds and backbone linkages.

In one embodiment, the nucleobase in an AON of the present invention is adenine, cytosine, guanine, thymine, or uracil. In another embodiment, the nucleobase is a modified form of adenine, cytosine, guanine, or uracil. In another embodiment, the modified nucleobase is hypoxanthine (the nucleobase in inosine), pseudouracil, pseudocytosine, 1-methylpseudouracil, orotic acid, agmatidine, lysidine, 2-thiouracil, 2-thiothymine, 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine, 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar. The term ‘nucleoside’ refers to the nucleobase linked to the (deoxy)ribosyl sugar. The term ‘nucleotide’ refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group, well known in the art), a nucleotide including a linker comprising a phosphodiester, phosphodiester, phosphoro(di)thioate, methylphosphonates, phosphoramidate linkers, and the like. Sometimes the terms adenosine and adenine, guanosine and guanine, cytosine and cytidine, uracil and uridine, thymine and thymidine, inosine and hypoxanthine, are used interchangeably to refer to the corresponding nucleobase, nucleoside or nucleotide. Sometimes the terms nucleobase, nucleoside and nucleotide are used interchangeably, unless the context clearly requires differently.

In one embodiment, an AON of the present invention comprises a 2′-substituted phosphorothioate monomer, preferably a 2′-substituted phosphorothioate RNA monomer, a 2′-substituted phosphate RNA monomer, or comprises 2′-substituted mixed phosphate/phosphorothioate monomers. It is noted that DNA is considered as an RNA derivative in respect of 2′ substitution. An AON of the present invention comprises at least one 2′-substituted RNA monomer connected through or linked by a phosphorothioate or phosphate backbone linkage, or a mixture thereof. The 2′-substituted RNA preferably is 2′-F, 2′-H (DNA), 2′-O-Methyl or 2′-O-(2-methoxyethyl). The 2′-O-Methyl is often abbreviated to “2′-OMe” and the 2′-O-(2-methoxyethyl) moiety is often abbreviated to “2′-MOE”. In a preferred embodiment of this aspect is provided an AON according to the invention, wherein the 2′-substituted monomer can be a 2′-substituted RNA monomer, such as a 2′-F monomer, a 2′-NH₂ monomer, a 2′-H monomer (DNA), a 2′-O-substituted monomer, a 2′-OMe monomer or a 2′-MOE monomer or mixtures thereof. Preferably, any other 2′-substituted monomer within the AON is a 2′-substituted RNA monomer, such as a 2′-OMe RNA monomer or a 2′-MOE RNA monomer, which may also appear within the AON in combination.

Throughout the application, a 2′-OMe monomer within an AON of the present invention may be replaced by a 2′-OMe phosphorothioate RNA, a 2′-OMe phosphate RNA or a 2′-OMe phosphate/phosphorothioate RNA. Throughout the application, a 2′-MOE monomer may be replaced by a 2′-MOE phosphorothioate RNA, a 2′-MOE phosphate RNA or a 2′-MOE phosphate/phosphorothioate RNA. Throughout the application, an oligonucleotide consisting of 2′-OMe RNA monomers linked by or connected through phosphorothioate, phosphate or mixed phosphate/phosphorothioate backbone linkages may be replaced by an oligonucleotide consisting of 2′-OMe phosphorothioate RNA, 2′-OMe phosphate RNA or 2′-OMe phosphate/phosphorothioate RNA. Throughout the application, an oligonucleotide consisting of 2′-MOE RNA monomers linked by or connected through phosphorothioate, phosphate or mixed phosphate/phosphorothioate backbone linkages may be replaced by an oligonucleotide consisting of 2′-MOE phosphorothioate RNA, 2′-MOE phosphate RNA or 2′-MOE phosphate/phosphorothioate RNA.

In addition to the specific preferred chemical modifications at certain positions in compounds of the invention, compounds of the invention may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e. the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-O-methyl, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino)propyl), 2′-O-(2-amino)ethyl, 2′-O-(2-(dimethylamino)ethyl); 2′-deoxy (DNA); 2′-O-(haloalkyl)methyl such as 2′-O-(2-chloroethoxy)methyl (MCEM), 2′4)-(2,2-dichloroethoxy)methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O-[2-(N,N-dimethylcarbamoyl)ethyl] (DOME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-O,4′-C) constrained ethyl (cEt) BNA monomer, a (2′-O,4′-C) constrained methoxyethyl (cMOE) BNA monomer, a 2′,4′-BNA^{NC}(NH) monomer, a 2′,4′-BNA^{NC}(NMe) monomer, a 2′,4′-BNA^{NC}(NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All of these modifications are known to the person skilled in the art.

A “backbone modification” indicates the presence of a modified version of the ribosyl moiety (“scaffold modification”), as indicated above, and/or the presence of a modified version of the phosphodiester as naturally occurring in RNA (“backbone linkage modification”). Examples of internucleoside linkage modifications are phosphorothioate (PS), chirally pure phosphorothioate, Rp phosphorothioate, Sp phosphorothioate, phosphorodithioate (PS2), phosphonoacetate (PACE), thophosphonoacetate, phosphonacetamide (PACA), thiophosphonacetamide, phosphorothioate prodrug, S-alkylated phosphorothioate, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphoryl guanidine (PGO), methylsulfonyl phosphoroamidate, phosphoramidite, phosphonamidite, phosphoramidate, N3′→P5′ thiophosphoramidate, phosphorodiamidate, phosphorothiodiamidate, sulfamate, dimethylenesulfoxide, sultanate, triazole, oxalyl, carbamate, methyleneimino (MMI), and thioacetamido (TANA); and their derivatives.

In a preferred aspect the AON of the present invention is an oligoribonucleotide. In a further preferred aspect, the AON according to the invention comprises at least one 2′-O alkyl modification, preferably a 2′-OMe modified sugar. In a more preferred embodiment, all nucleotides in said AON are 2′-OMe modified. In another preferred aspect, the invention relates to an AON comprising at least one 2′-MOE modification. In a more preferred embodiment, all nucleotides of said AON carry a 2′-MOE modification. In yet another aspect the invention relates to an AON, wherein the AON comprises at least one 2′-OMe and at least one 2′-MOE modification. Preferably, the AON according to the present invention has at least one non-naturally occurring internucleoside linkage. A preferred non-naturally occurring internucleoside modification is a modification with phosphorothioate (a phosphorothioate linkage). In a more preferred aspect, all sequential nucleotides of the AON of the present invention are interconnected by phosphorothioate linkages.

In yet another aspect, the invention relates to a pharmaceutical composition comprising an AON according to the invention, and a pharmaceutically acceptable carrier. Preferably, the pharmaceutical composition is for intravitreal administration and is dosed in an amount ranging from about 0.01 mg to about 1 mg of total AON per eye. More preferably, the pharmaceutical composition is for intravitreal administration and is dosed in an effective amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 μg total AON per eye (generally using a higher loading dose to reach the effective amount). Often a regime is chosen in which the loading dose (the first dose that a patient receives) is higher than the follow-up doses. In a preferred setting, the loading dose is twice the amount of each of the follow-up doses. Non-limiting examples of such follow-up doses are for instance a 40 μg dose (with a loading dose of 80 μg) or a 80 μg dose (with a loading dose of 160 μg) per eye, as used in a clinical set-up in the treatment of LCA10 caused by the c.2991+1655A>G mutation, in which the aberrant 128 bp exon needed to be skipped using a 17-mer oligonucleotide. Clearly, depending on the size of the active compound (number of nucleotides and content) as well as the effectiveness of the skip, such dosage amounts and regimens can change.

In another embodiment, the invention relates to a viral vector expressing an AON according to the invention. In another embodiment, the invention relates to an AON according to the invention, a pharmaceutical composition according to the invention, or a viral vector according to the invention, for use as a medicament. In another embodiment, the invention relates to an AON according to the invention, a pharmaceutical composition according to the invention, or a viral vector according to the invention, for treatment, prevention or delay of a CEP290-related disease or a condition requiring modulating splicing of CEP290 pre-mRNA, such as LCA10. More preferably, the invention relates to an AON according to the invention, a pharmaceutical composition according to the invention, or a viral vector according to the invention, for the treatment, prevention or delay of LCA10, caused by a mutation in exon 36 of the CEP290 gene. More preferably, the mutation causing said LCA10 is the c.4723A>T mutation, introducing a premature stop codon in the CEP290 transcript.

The invention also relates to a use of an AON according to the invention, a pharmaceutical composition according to the invention, or a viral vector according to the invention for the preparation of a medicament. Preferably, said medicament is for treatment, prevention or delay of a CEP290-related disease or condition requiring modulating splicing of CEP290 pre-mRNA, such as LCA10. In yet another aspect, the invention relates to a use of an AON according to the invention, a pharmaceutical composition according to the invention, or a viral vector according to the invention, for the preparation of a medicament for the treatment, prevention or delay of LCA10, caused by a mutation in exon 36 of the CEP290 gene. More preferably, the mutation causing said LCA10 is the c.4723A>T mutation, introducing a premature stop codon in the CEP290 transcript.

The present invention also relates to a method for modulating splicing of CEP290 pre-mRNA in a cell, said method comprising contacting said cell with an AON according to the invention, a pharmaceutical composition according to the invention, or a viral vector according to the invention. In a preferred embodiment, the invention relates to a method for the treatment of an individual suffering from LCA10, said method comprising contacting a cell of said individual with an AON according to the invention, a pharmaceutical composition according to the invention, or a viral vector according to the invention.

The present invention, in another embodiment, relates to a method of treating LCA10 patients, wherein said method comprises administering a loading dose that is preferably higher, and more preferably twice the amount of the follow-up dose, and wherein said method comprises administering 1, 2, 3, 4, 5, 6, 7, 8, 9, 10, or 11 follow-up doses per year, and wherein the follow-up dose is selected from the group consisting of: 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 μg total AON per eye. In one embodiment, the AON can be administered as is, or naked, whereas in yet another embodiment the AON is delivered through a delivery vehicle, preferably an adenovirus associated virus, or AAV vector. The AON, when administered as is, or through a delivery vehicle, is administered into the vitreous by injection, to yield exon skipping in photoreceptor cells where the CEP290 protein acts.

In all embodiments of the invention, the terms ‘modulating splicing’ and ‘exon skipping’ are synonymous. In respect of CEP290, ‘modulating splicing’ or ‘exon skipping’ are herein to be construed as the exclusion of (mutated) exon 36 from the human CEP290 transcript.

The term ‘exon skipping’ is herein defined as inducing, producing or increasing production within a cell of a mature mRNA that does not contain a particular exon (in the current case exon 36 of the CEP290 gene) that would be present in the mature mRNA without exon skipping. Exon skipping is achieved by providing a cell expressing the pre-mRNA of said mature mRNA with a molecule capable of interfering with sequences such as, for example, the (cryptic) splice donor or (cryptic) splice acceptor sequence required for allowing the enzymatic process of splicing, or with a molecule that is capable of interfering with an exon inclusion signal required for recognition of a stretch of nucleotides as an exon to be included in the mature mRNA; such molecules are herein referred to as ‘exon skipping molecules’, as ‘exon 36 skipping molecules’, as ‘exon skipping AONs’, or as ‘AONs capable of skipping exon 36 from human CEP290 pre-mRNA’, or as ‘AONs capable of reducing the inclusion of exon 36 in human CEP290 mRNA’. The term ‘pre-mRNA’ refers to a non-processed or partly processed precursor mRNA that is synthesized from a DNA template of a cell by transcription, such as in the nucleus. The term ‘mRNA’ refers to a processed RNA molecule that is translated to a protein in the cytoplasm of the cell, preferably, according to the present invention, lacking exon 36 when it concerns a CEP290 mRNA derived from a mutant CEP290 gene carrying a mutated exon 36.

The term ‘antisense oligonucleotide’ (AON) is understood to refer to a nucleotide sequence which is substantially complementary to a (target) nucleotide sequence in a gene, a pre-mRNA molecule, hnRNA (heterogenous nuclear RNA) or mRNA molecule. The degree of complementarity (or substantial complementarity) of the antisense sequence is preferably such that a molecule comprising the antisense sequence can form a stable double stranded hybrid with the target nucleotide sequence in the (pre-)mRNA molecule under physiological conditions. The terms ‘AON’, ‘antisense oligonucleotide’, ‘oligonucleotide’ and ‘oligo’ are used interchangeably herein and are understood to refer to an oligonucleotide comprising an antisense sequence in respect of the target sequence.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The word “about” or “approximately” when used in association with a numerical value (e.g. about 10 μg) preferably means that the value may be the given value (of 10 μg)±0.1% of the value.

In one embodiment, an exon 36 skipping molecule as defined herein is an AON that binds and/or is complementary to a specified sequence. Binding to one of the specified target sequences, preferably in the context of the mutated CEP290 exon 36 may be assessed via techniques known to the skilled person. A preferred technique is a gel mobility shift assay as described in EP1619249. In a preferred embodiment, an exon 36 skipping AON is said to bind to one of the specified sequences as soon as a binding of said molecule to a labeled target sequence is detectable in a gel mobility shift assay.

In all embodiments of the invention, an exon 36 skipping molecule is preferably an AON. Preferably, an exon 36 skipping AON according to the invention is an AON, which comprises a sequence that is complementary or substantially complementary to a nucleotide sequence as shown in SEQ ID NO:42, 44, 46, 48 or 147.

The term ‘substantially complementary’ used in the context of the invention indicates that some mismatches in the antisense sequence are allowed as long as the functionality, i.e. inducing skipping of the mutated CEP290 exon 36 is still acceptable. Preferably, the complementarity is from 90% to 100%. In general, this allows for 1 or 2 mismatches in an AON of 20 nucleotides or 1, 2, 3 or 4 mismatches in an AON of 40 nucleotides, or 1, 2, 3, 4, 5, or 6 mismatches in an AON of 60 nucleotides, etc. As can be seen in the accompanying non-limiting examples below, exon 36 skipping in wild type (non-mutated) CEP290 pre-mRNA was observed when an AON was used (e.g. QRX136.30) that is not 100% complementary to the wild type sequence, but in fact is 100% complementary to a sequence overlapping the c.4723A>T mutation in the exon 36 sequence (1 mismatch in a 22-mer oligonucleotide).

The invention provides a method for designing an exon 36 skipping AON able to induce skipping of exon 36 of the human CEP290 pre-mRNA. First, said AON is selected to bind to and/or to be complementary to exon 36, possibly with stretches of the flanking intron sequences as shown in SEQ ID NO:1 (exon 36 with c.4723A>T mutation+surrounding sequences of intron 35 and intron 36, at the 5′ and 3′ ends of the exon, respectively) and as shown in SEQ ID NO:2 (wild type exon 36+surrounding sequences of intron 35 and intron 36). It is to be understood, that although SEQ ID NO:1 to 4 display DNA sequences, these also represent their respective RNA sequences, when transcribed into pre-mRNA and subsequently mRNA. The pre-mRNA is the preferred target molecule for the AONs of the present invention.

In a preferred method at least one of the following aspects has to be taken into account for designing, improving said exon skipping AON further: the exon skipping AON preferably does not contain a CpG island or a stretch of CpG islands; and the exon skipping AON has acceptable RNA binding kinetics and/or thermodynamic properties. The presence of a CpG or a stretch of CpG in an AON is usually associated with an increased immunogenicity of said AON. This increased immunogenicity is undesired since it may induce damage of the tissue to be treated, i.e. the eye. Immunogenicity may be assessed in an animal model by assessing the presence of CD4+ and/or CD8+ cells and/or inflammatory mononucleocyte infiltration. Immunogenicity may also be assessed in blood of an animal or of a human being treated with an AON of the invention by detecting the presence of a neutralizing antibody and/or an antibody recognizing said AON using a standard immunoassay known to the skilled person. An inflammatory reaction, type I-like interferon production, IL-12 production and/or an increase in immunogenicity may be assessed by detecting the presence or an increasing amount of a neutralizing antibody or an antibody recognizing said AON using a standard immunoassay. The RNA binding kinetics and/or thermodynamic properties are at least in part determined by the melting temperature of an AON (Tm; calculated with an oligonucleotide properties calculator known to the person skilled in the art), and/or the free energy of the AON-target exon complex. If a Tm is too high, the AON is expected to be less specific. An acceptable Tm and free energy depend on the sequence of the AON. Therefore, it is difficult to give preferred ranges for each of these parameters. An acceptable Tm may be ranged between 35 and 70° C. and an acceptable free energy may be ranged between 15 and 45 kcal/mol.

An AON of the invention is preferably one that can exhibit an acceptable level of functional activity. A functional activity of said AON is preferably to induce the skipping of exon 36 from CEP290 pre-mRNA (or in other words, to reduce the inclusion of exon 36 in CEP290 mRNA) to a certain acceptable level, to provide an individual with a functional CEP290 protein and/or at least in part decreasing the production of a prematurely terminated CEP290 protein. In a preferred embodiment, an AON is said to be capable of inducing skipping of CEP290 exon 36, when the CEP290 exon 36 skipping percentage as measured by real-time quantitative RT-PCR analysis or digital droplet PCR (ddPCR) is at least 2-10%, preferably at least 10-20%, more preferably at least 20-30%, even more preferably at least 30-40%, and most preferably at least 45%, at least 50%, at least 55%, at least 60%, at least 65%, at least 70%, at least 75%, at least 80%, at least 85%, at least 90%, at least 95%, or 100% as compared to a control RNA product not treated with an AON or a negative control AON. The present disclosure now enables the skilled person to generate an AON that provides significant levels of exon 36 skipping from CEP290 pre-mRNA. It is to be understood that when AONs become too short (such that they become non-specific for the target sequence), or too long (such that they can no longer enter the cell, aggregate and/or become degraded), even though they are complementary to (a part of) the exon 36 sequences+/−its surrounding sequences, that they would not be considered part of the invention if they are incapable of providing exon 36 skipping from the human CEP290 pre-mRNA, with the percentages given above, and as outlined in detail herein.

An AON that comprises a sequence that is complementary or substantially complementary to a nucleotide sequence as shown in SEQ ID NO:1, 2, 3, or 4 (as RNA) of CEP290 is such that the (substantially) complementary part is at least 50% of the length of the AON according to the invention, more preferably at least 60%, even more preferably at least 70%, even more preferably at least 80%, even more preferably at least 90% or even more preferably at least 95%, or even more preferably 98% or even more preferably at least 99%, and most preferably 100% to a stretch of consecutive nucleotides in the target sequence. Preferably, an AON according to the invention comprises or consists of a sequence that is complementary to part of SEQ ID NO:1, 2, 3, or 4 (or in fact their (pre-) mRNA equivalents).

In another preferred embodiment, the length of said complementary part of said AON is at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. More preferably, the length of said complementarity is 15, 16, 17, 18, 19, 20, 21, 22, 23, 24, 25, 26, 27, 28, 29, or 30 nucleotides. Even more preferably, the length of said complementarity is 15, 16, 17, 18, 19, 20, 21, 22, 23, 24, or 25 nucleotides. Most preferably, the target sequence is 15, 16, 17, 18, 19, or 20 consecutive nucleotides in the sequence represented by SEQ ID NO:147. From an AON side, the preferred length of an AON according to the invention is at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. Preferably, the length of an AON according to the invention is 15, 16, 17, 18, 19, 20, 21, 22, 23, 24, 25, 26, 27, 28, 29, or 30 nucleotides. More preferably, the length of an AON according to the invention is 15, 16, 17, 18, 19, 20, 21, 22, 23, 24, or 25 nucleotides. Most preferably, the AON consists of 15, 16, 17, 18, 19, or 20 nucleotides that are 100% complementary to a consecutive stretch of nucleotides in SEQ ID NO:147.

Additional flanking sequences may be used to modify the binding of a protein to the AON, or to modify a thermodynamic property of the AON, more preferably to modify target RNA binding affinity. It is thus not absolutely required that all the bases in the region of complementarity are capable of pairing with bases in the opposing strand. For instance, when designing the AON, one may want to incorporate for instance a residue that does not base pair with the base on the complementary strand. Mismatches may, to some extent, be allowed, if under the circumstances in the cell, the stretch of nucleotides is sufficiently capable of hybridizing to the complementary part. In this context, ‘sufficiently’ preferably means that in a gel mobility shift assay as noted above, binding of an AON is detectable.

Optionally, said AON may further be tested by transfection into retina cells of patients, or in optic cups generated form patient material, as exemplified herein. Skipping of targeted exon 36 may be assessed by RT-PCR (such as e.g. described in EP1619249 and WO 2016/005514) or ddPCR as described herein. The complementary regions are preferably designed such that, when combined, they are specific for the exon or the exon/intron region in the pre-mRNA. Such specificity may be created with various lengths of complementary regions as this depends on the actual sequences in other (pre-) mRNA molecules in the system. The risk that the AON also will be able to hybridize to one or more other pre-mRNA molecules decreases with increasing size of the AON. It is clear that AONs comprising mismatches in the region of complementarity but that retain the capacity to hybridize and/or bind to the targeted region(s) in the pre-mRNA, can be used in the invention. However, preferably at least the complementary parts do not comprise such mismatches as AONs lacking mismatches in the complementary part typically have a higher efficiency and a higher specificity, than AONs having such mismatches in one or more complementary regions. It is thought that higher hybridization strengths (i.e. increasing number of interactions with the opposing strand) are favorable in increasing the efficiency of the process of interfering with the splicing machinery of the system. An exon skipping AON of the invention is, when produced, preferably an isolated single stranded molecule in the absence of its (target) counterpart sequence.

An exon 36 skipping AON according to the invention preferably contains all ribonucleosides, which are preferably substituted at the 2′ position of the sugar moiety (to render the oligonucleotide more resistant to nuclease breakdown and to increase its targeting efficiency). Uridines in an AON according to the invention may be 5-methyluridine, or just uridine without a 5-methyl group in the base. Similarly, cytidines in an AON according to the invention may be 5-methylcytidine, or just cytidine without a 5-methyl group in the base. An AON according to the invention may contain one of more DNA residues, and/or one or more nucleotide analogues or equivalents. It is preferred that an exon 36 skipping AON of the invention comprises one or more residues that are modified to increase nuclease resistance, and/or to increase the affinity of the AON for the target sequence. Therefore, in a preferred embodiment, the AON sequence comprises at least one nucleotide analogue or equivalent, wherein a nucleotide analogue or equivalent is defined as a residue having a modified base, and/or a modified backbone, and/or a non-naturally occurring internucleoside linkage, or a combination of these modifications. Most preferably, all internucleoside linkages are modified to render the oligonucleotide more resistant to breakdown, and all sugar moieties of the nucleosides are substituted at the 2′, 3′ and/or 5′ position, to render the oligonucleotide more resistant to breakdown.

In a preferred embodiment, the nucleotide analogue or equivalent comprises a modified backbone. Examples of such backbones are morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery. However, because the morpholino backbone is uncharged, cationic lipids are not effective mediators of morpholino oligonucleotide uptake in cells.

It is further preferred that the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A particular nucleotide analogue or equivalent that may be applied comprises a Peptide Nucleic Acid (PNA), having a modified polyamide backbone. PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer. Since the backbone of a PNA molecule contains no charged phosphate groups, PNA-RNA hybrids are usually more stable than RNA-RNA or RNA-DNA hybrids, respectively.

It is understood by a skilled person that it is not necessary for all positions in an AON to be modified uniformly. In addition, more than one of the analogues or equivalents as outlined herein may be incorporated in a single AON or even at a single position within an AON. In certain embodiments, an AON of the invention has at least two different types of analogues or equivalents. A preferred exon skipping AON according to the invention is a 2′-O alkyl phosphorothioated antisense oligonucleotide, such as an AON comprising a 2′-O-methyl modified ribose, a 2′-O-ethyl modified ribose, a 2′-O-propyl modified ribose, and/or substituted derivatives of these modifications such as halogenated derivatives. An effective AON according to the invention comprises a 2′-OMe ribose with a (preferably full) phosphorothioated backbone. Another preferred exon skipping AON according to the invention is a 2′-methoxyethoxy phosphorothioated antisense oligonucleotide (an AON comprising 2′-MOE modified riboses, and/or substituted derivatives of these modifications such as halogenated derivatives). An effective AON according to the invention comprises a 2′-MOE ribose with a (preferably full) phosphorothioated backbone.

It will also be understood by a skilled person that different AONs can be combined for efficiently skipping of the mutant CEP290 exon 36, as exemplified herein. In a preferred aspect, a combination of at least two AONs are used in a method of the invention, such as 2, 3, 4, or 5 different AONs. Hence, the invention also relates to a set of AONs comprising at least one AON according to the present invention.

An AON can be linked to a moiety that enhances uptake of the AON in cells, preferably retina or photoreceptor cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain.

An exon 36 skipping AON according to the invention may be indirectly administrated using suitable means known in the art. It may for example be provided to an individual or a cell, tissue or organ of said individual in the form of an expression vector wherein the expression vector encodes a transcript comprising said oligonucleotide. The expression vector is preferably introduced into a cell, tissue, organ or individual via a gene delivery vehicle. In a preferred embodiment, there is provided a viral-based expression vector comprising an expression cassette or a transcription cassette that drives expression or transcription of an AON as identified herein. Accordingly, the invention provides a viral vector expressing an exon 36 skipping AON according to the invention when placed under conditions conducive to expression of the exon skipping AON. A cell can be provided with an exon skipping molecule capable of interfering with essential sequences that result in highly efficient skipping of the mutated CEP290 exon 36 by plasmid-derived AON expression or viral expression provided by adenovirus- or adeno-associated virus-based vectors. Expression may be driven by a polymerase II-promoter (Pol II) such as a U7 promoter or a polymerase III (Pol III) promoter, such as a U6 RNA promoter. A preferred delivery vehicle is AAV, or a retroviral vector such as a lentivirus vector and the like. Also, plasmids, artificial chromosomes, plasmids usable for targeted homologous recombination and integration in the human genome of cells may be suitably applied for delivery of an oligonucleotide as defined herein. Preferred for the current invention are those vectors wherein transcription is driven from Pol III promoters, and/or wherein transcripts are in the form fusions with U1 or U7 transcripts, which yield good results for delivering small transcripts. It is within the skill of the artisan to design suitable transcripts. Preferred are Pal III driven transcripts, preferably, in the form of a fusion transcript with an U1 or U7 transcript, known to the person skilled in the art.

Typically, when the exon 36 skipping AON is delivered by a viral vector, it is in the form of an RNA transcript that comprises the sequence of an oligonucleotide according to the invention in a part of the transcript. An MV vector according to the invention is a recombinant AAV vector and refers to an MV vector comprising part of an AAV genome comprising an encoded exon 36 skipping AON according to the invention encapsidated in a protein shell of capsid protein derived from an AAV serotype. Part of an AAV genome may contain the inverted terminal repeats (ITR) derived from an adeno-associated virus serotype, such as AAV1, AAV2, AAV3, AAV4, AAV5, AAV6, AAV7, AAV8, AAV9 and others. Protein shell comprised of capsid protein may be derived from an MV serotype such as AAV1, 2, 3, 4, 5, 6, 7, 8, 9 and others. A protein shell may also be named a capsid protein shell. MV vector may have one or preferably all wild type AAV genes deleted but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, if they remain functional. In this context, functionality refers to the ability to direct packaging of the genome into the capsid shell and then allow for expression in the host cell to be infected or target cell. In the context of the invention a capsid protein shell may be of a different serotype than the MV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV2 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention. Preferably, a recombinant AAV vector according to the invention comprises a capsid protein shell of AAV serotype 2, 5, 8 or AAV serotype 9 wherein the AAV genome or ITRs present in said AAV vector are derived from AAV serotype 2, 5, 8 or MV serotype 9; such MV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

More preferably, a recombinant AAV vector according to the invention comprises a capsid protein shell of AAV serotype 2 and the AAV genome or ITRs present in said vector are derived from AAV serotype 5; such vector is referred to as an AAV 2/5 vector. More preferably, a recombinant AAV vector according to the invention comprises a capsid protein shell of MV serotype 2 and the AAV genome or ITRs present in said vector are derived from AAV serotype 8; such vector is referred to as an AAV 2/8 vector. More preferably, a recombinant AAV vector according to the invention comprises a capsid protein shell of MV serotype 2 and the AAV genome or ITRs present in said vector are derived from AAV serotype 9; such vector is referred to as an AAV 2/9 vector. More preferably, a recombinant AAV vector according to the invention comprises a capsid protein shell of MV serotype 2 and the AAV genome or ITRs present in said vector are derived from AAV serotype 2; such vector is referred to as an MV 2/2 vector. A nucleic acid molecule encoding an exon 36 skipping AON according to the invention represented by a nucleic acid sequence of choice is preferably inserted between the MV genome or ITR sequences as identified above, for example an expression construct comprising an expression regulatory element operably linked to a coding sequence and a 3′ termination sequence. “AAV helper functions” generally refers to the corresponding AAV functions required for AAV replication and packaging supplied to the MV vector in trans. AAV helper functions complement the AAV functions which are missing in the MV vector, but they lack MV ITRs (which are provided by the AAV vector genome). AAV helper functions include the two major ORFs of AAV, namely the rep coding region and the cap coding region or functional substantially identical sequences thereof. Rep and Cap regions are well known in the art. The AAV helper functions can be supplied on an AAV helper construct, which may be a plasmid.

Introduction of the helper construct into the host cell can occur e.g. by transformation, transfection, or transduction prior to or concurrently with the introduction of the AAV genome present in the AAV vector as identified herein. The AAV helper constructs of the invention may thus be chosen such that they produce the desired combination of serotypes for the AAV vector's capsid protein shell on the one hand and for the MV genome present in said MV vector replication and packaging on the other hand. “AAV helper virus” provides additional functions required for MV replication and packaging.

Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456. Preferably, an MV genome as present in a recombinant AAV vector according to the invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gip) or a gene encoding a chemically, enzymatically or otherwise detectable and/or selectable product (e.g. lacZ, aph, etc.) known in the art. A preferred AAV vector according to the invention is an MV vector, preferably an AAV2/5, AAV2/8, AAV2/9 or AAV2/2 vector, expressing an CEP290 exon 36 skipping AON according to the invention that comprises, or preferably consists of, a sequence that is complementary or substantially complementary to a nucleotide sequence as shown in SEQ ID NO:42, 44, 46, 48, or 147. In another aspect, the AAV vector according to the present invention encodes an AON comprising or consisting of a sequence selected from the group consisting of: SEQ ID NO:7, 8, 11, 12, 15, 16, 18, 19, 26, 27, 28, 29, 37, 38, 39, 40, 41, 51, 52, 53, 54, 55, 56, 57, 58, 59, 60, 61, 63, 64, 65, 66, 67, 70, 71, 72, 74, 75, 76, 77, 78, and 93 to 146. In another aspect, the AAV vector according to the present invention encodes an AON comprising or consisting of a sequence selected from the group consisting of: SEQ ID NO:7, 8, 12, 19, 26, 27, 28, 29, 39, 53, 54, 55, 56, 57, 58, 60, 61, 74, 75, 76, 77, 78, and 93 to 146. In another aspect, the AAV vector according to the present invention encodes an AON comprising or consisting of a sequence selected from the group consisting of: SEQ ID NO:53, 54, 55, 56, 57, 58, 61, 74, 75, 76, 77, 78, and 93 to 146. In another aspect, the AAV vector according to the present invention encodes an AON comprising or consisting of a sequence selected from the group consisting of: SEQ ID NO:53, 54, 55, 56, 58, 74, 75, 76, 77, 78, 105, 106, 125, 126, 143, 144, 145, and 146. A further preferred AAV vector according to the invention is an AAV vector, preferably an AAV2/5, AAV2/8, AAV2/9 or AAV2/2 vector, expressing an exon 36 skipping AON according to the invention.

An exon 36 skipping AON according to the invention can be delivered as is to an individual, a cell, tissue or organ of said individual. When administering an exon 36 skipping AON according to the invention, it is preferred that the AON is dissolved in a solution that is compatible with the delivery method. Preferably viral vectors or nanoparticles are delivered to retina cells. Such delivery to retina cells or other relevant cells may be in vivo, in vitro or ex vivo. Nanoparticles and micro particles that may be used for in vivo AON delivery are well known in the art. Alternatively, a plasmid can be provided by transfection using known transfection reagents. For intravenous, subcutaneous, intramuscular, intrathecal and/or intraventricular administration it is preferred that the solution is a physiological salt solution. Particularly preferred in the invention is the use of an excipient or transfection reagents that will aid in delivery of each of the constituents as defined herein to a cell and/or into a cell (preferably a retina cell). Preferred are excipients or transfection reagents capable of forming complexes, nanoparticles, micelles, vesicles and/or liposomes that deliver each constituent as defined herein, complexed or trapped in a vesicle or liposome through a cell membrane. Many of these excipients are known in the art. Suitable excipients or transfection reagents comprise polyethylenimine (PEI; ExGen500 (MBI Fermentas)), LipofectAMINE™ 2000 (Invitrogen) or derivatives thereof, or similar cationic polymers, including polypropyleneimine or polyethylenimine copolymers (PECs) and derivatives, synthetic amphiphils (SAINT-18), Lipofectin™, DOTAP and/or viral capsid proteins that are capable of self-assembly into particles that can deliver each constituent as defined herein to a cell, preferably a retina cell. Such excipients have been shown to efficiently deliver an AON to a wide variety of cultured cells, including retina cells. Their high transfection potential is combined with an excepted low to moderate toxicity in terms of overall cell survival. The ease of structural modification can be used to allow further modifications and the analysis of their further (in vivo) nucleic acid transfer characteristics and toxicity. Lipofectin represents an example of a liposomal transfection agent. It consists of two lipid components, a cationic lipid N-[1-(2,3 dioleoyloxy)propyl]-N, N, N-trimethylammonium chloride (DOTMA) (cp. DOTAP which is the methylsulfate salt) and a neutral lipid dioleoylphosphatidyl ethanolamine (DOPE). The neutral component mediates the intracellular release. Another group of delivery systems are polymeric nanoparticles. Polycations such as diethylamino ethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver AONs across cell membranes into cells. In addition to these common nanoparticle materials, the cationic peptide protamine offers an alternative approach to formulate an oligonucleotide with colloids. This colloidal nanoparticle system can form so called proticles, which can be prepared by a simple self-assembly process to package and mediate intracellular release of an AON. The skilled person may select and adapt any of the above or other commercially available alternative excipients and delivery systems to package and deliver an exon skipping molecule for use in the current invention to deliver it for the prevention, treatment or delay of a CEP290-related disease or condition.

“Prevention, treatment or delay of a CEP290-related disease or condition” is herein preferably defined as preventing, halting, ceasing the progression of, or reversing partial or complete visual impairment or blindness that is caused by a genetic defect in the CEP290 gene.

In addition, an exon 36 skipping AON according to the invention could be covalently or non-covalently linked to a targeting ligand specifically designed to facilitate the uptake into the cell, cytoplasm and/or its nucleus. Such ligand could comprise (i) a compound (including but not limited to peptide(-like) structures) recognizing cell, tissue or organ specific elements facilitating cellular uptake and/or (ii) a chemical compound able to facilitate the uptake in to cells and/or the intracellular release of an oligonucleotide from vesicles, e.g. endosomes or lysosomes. Therefore, in a preferred embodiment, an exon 36 skipping AON according to the invention is formulated in a composition or a medicament or a composition, which is provided with at least an excipient and/or a targeting ligand for delivery and/or a delivery device thereof to a cell and/or enhancing its intracellular delivery.

It is to be understood that if a composition comprises an additional constituent such as an adjunct compound as later defined herein, each constituent of the composition may not be formulated in one single combination or composition or preparation. Depending on their identity, the skilled person will know which type of formulation is the most appropriate for each constituent as defined herein. In a preferred embodiment, the invention provides a composition or a preparation which is in the form of a kit of parts comprising an exon 36 skipping AON according to the invention and a further adjunct compound as later defined herein. If required, an exon 36 skipping AON according to the invention or a vector, preferably a viral vector, expressing an exon 36 skipping AON according to the invention can be incorporated into a pharmaceutically active mixture by adding a pharmaceutically acceptable carrier. Accordingly, the invention also provides a composition, preferably a pharmaceutical composition, comprising an exon 36 skipping AON according to the invention, or a viral vector according to the invention and a pharmaceutically acceptable excipient. Such composition may comprise a single exon 36 skipping AON or viral vector according to the invention, but may also comprise multiple, distinct exon 36 skipping AON or viral vectors according to the invention. Such a pharmaceutical composition may comprise any pharmaceutically acceptable excipient, including a carrier, filler, preservative, adjuvant, solubilizer and/or diluent. Such pharmaceutically acceptable carrier, filler, preservative, adjuvant, solubilizer and/or diluent may for instance be found in Remington (Remington. 2000. The Science and Practice of Pharmacy, 20th Edition. Baltimore, Md.: Lippincott Williams Wilkins). Each feature of said composition has earlier been defined herein. A preferred route of administration is through intra-vitreal injection of an aqueous solution or specially adapted formulation for intraocular administration. EP2425814 discloses an oil in water emulsion especially adapted for intraocular (intravitreal) administration of peptide or nucleic acid drugs. This emulsion is less dense than the vitreous fluid, so that the emulsion floats on top of the vitreous, avoiding that the injected drug impairs vision.

The invention relates to AON capable of inducing skipping exon 36 from human CEP290 pre-mRNA, wherein the AON comprises a sequence that is substantially complementary to a sequence of exon 36 of the human CEP290 gene or a part thereof, or wherein the AON comprises a sequence that is substantially complementary to a sequence of exon 36 of the human CEP290 gene or a part thereof and overlaps with the exon 36/intron 36 boundary at the 3′ end of exon 36 and the 5′ end of intron 36. In a preferred embodiment, the AON comprises or consists of a sequence that is substantially complementary to a sequence selected from the group consisting of: SEQ ID NO:42, 44, 46, 48, and 147. In another preferred embodiment, the AON consists of 15, 16, 17, 18, 19, or 20 nucleotides that are 100% complementary to a consecutive sequence within SEQ ID NO:147. In yet another preferred embodiment, the AON comprises or consists of a sequence selected from the group consisting of: SEQ ID NO:7, 8, 11, 12, 15, 16, 18, 19, 26, 27, 28, 29, 37, 38, 39, 40, 41, 51, 52, 53, 54, 55, 56, 57, 58, 59, 60, 61, 63, 64, 65, 66, 67, 70, 71, 72, 74, 75, 76, 77, 78, and 93 to 146. In a more preferred embodiment, the AON consists of a sequence selected from the group consisting of: SEQ ID NO:53, 54, 55, 56, 57, 58, 61, 74, 75, 76, 77, 78, and 93 to 146. In an even more preferred embodiment, the AON consists of a sequence selected from the group consisting of: SEQ ID NO:53, 54, 55, 56, 58, 74, 75, 76, 77, 78, 105, 106, 125, 126, 143, 144, 145, and 146. In another preferred embodiment, the AON according to the invention comprises at least one sugar moiety carrying a 2′-OMe modification or a 2′-MOE modification. In one preferred embodiment, all nucleosides within the AON are 2′-OMe modified. In yet another preferred embodiment, all nucleosides within the AON are 2′-MOE modified.

The present invention also relates to a pharmaceutical composition comprising an AON according to the invention, and a pharmaceutically acceptable carrier. Pharmaceutically acceptable carriers may be selected from a wide variety of solvents, carriers, etc. well known to the person skilled in the art. Preferably, the pharmaceutical composition of the present invention is for intravitreal administration and is dosed in an amount ranging from 0.01 mg and 1 mg of total AON per eye. More preferably, the pharmaceutical composition is for intravitreal administration and is dosed in an amount ranging from 0.1 and 1 mg of total AON per eye, such as about 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 μg total AON per eye.

In another aspect, the invention relates to a viral vector expressing an AON according to the invention. Preferred viral vectors that may be applied for such purposes are disclosed above.

In another aspect, the invention relates to an AON according to the invention, a pharmaceutical composition according to the invention, or a viral vector according to the invention, for use as a medicament.

In yet another aspect, the invention relates to an AON according to the invention, a pharmaceutical composition according to the invention, or a viral vector according to the invention, for treatment, prevention or delay of a CEP290-related disease or a condition requiring modulating splicing of human CEP290 pre-mRNA, such as Leber's Congenital Amaurosis type 10 (LCA10). More preferably, the LCA10 treatment is in patients carrying exon 36 mutations, and who would benefit from a skip of exon 36 to subsequently obtain a full length CEP290 protein in their affected retinal cells.

In yet another embodiment, the invention relates to a use of an AON according to the invention, a pharmaceutical composition according to the invention, or a viral vector according to the invention for the preparation of a medicament for the treatment, prevention or delay of a CEP290-related disease or condition requiring modulating splicing of CEP290 pre-mRNA. Preferably, the CEP290-related disease is LCA10.

The invention also relates to a method for modulating splicing of CEP290 pre-mRNA in a cell, said method comprising contacting said cell with an AON according to the invention, a pharmaceutical composition according to the invention, or a viral vector according to the invention.

In another embodiment, the invention relates to a method for the treatment of a CEP290 related disease or condition requiring modulating splicing of CEP290 pre-mRNA of an individual in need thereof, said method comprising the step of contacting a cell of said individual with an AON according to the invention, a pharmaceutical composition according to the invention, or a viral vector according to the invention. The methods of the invention preferably further comprise the step of assessing whether exon 36 skip has taken place in the cell or cells that have been treated with the AON according to the invention. Preferred regimens and dosages for methods of treatment are as disclosed herein.

SEQUENCES (5′ to 3′)
SEQ ID NO: 1 (exon 36 with c.4723A>T mutation +
surrounding sequences):
ATAGTTTCGATTTTCTGTAAAACAGGAGCAAAGAGAAATTG
TGTAGAAACATGAGGAAGACCTTCATATTCTTCATCACAGA
TTAGAACTACAGGCTGATAGTTCACTAAATAAATTCAAACA
AACGGCTTGGGTAAGATTCTAAGAACTTTGTTCCA
SEQ ID NO: 2 (wild type exon 36
surrounding sequences):
ATAGTTTCGATTTTCTGTAAAACAGGAGCAAAGAGAAATT
GTGAAGAAACATGAGGAAGACCTTCATATTCTTCATCACA
GATTAGAACTACAGGCTGATAGTTCACTAAATAAATTCAA
ACAAACGGCTTGGGTAAGATTCTAAGAACTTTGTTCCA
SEQ ID NO: 3 (exon 36 with c.4723A>T mutation):
GAGCAAAGAGAAATTGTGTAGAAACATGAGGAAGACCTTC
ATATTCTTCATCACAGATTAGAACTACAGGCTGATAGTTC
ACTAAATAAATTCAAACAAACGGCTTGG
SEQ ID NO: 4 (wild type exon 36):
GAGCAAAGAGAAATTGTGAAGAAACATGAGGAAGACCTTC
ATATTCTICATCACAGATTAGAACTACAGGCTGATAGTTC
ACTAAATAAATTCAAACAAACGGCTTGG
SEQ ID NO: 5 (AON QRX136.27):
CCUGUUUUACAGAAAAUCGAAA
SEQ ID NO: 6 (AON QRX136.28):
CUUUGCUCCUGUUUUACAGAAA
SEQ ID NO: 7 (AON QRX136.29 and QRX136.29a):
CUCUUUGCUCCUGUUUUACAG
SEQ ID NO: 8 (AON QRX136.30):
CUACACAAUUUCUCUUUGCUCC
SEQ ID NO: 9 (AON QRX136.31):
UUCUACACAAUUUCUCUUUGC
SEQ ID NO: 10 (AON QRX136.32):
CAUGUUUCUACACAAUUUCUC
SEQ ID NO: 11 (AON QRX136.33):
CUUCCUCAUGUUUCUACACAA
SEQ ID NO: 12 (AON QRX136.34):
GAAGGUCUUCCUCAUGUUUCUA
SEQ ID NO: 13 (AON QRX136.35):
GAAUAUGAAGGUCUUCCUCAU
SEQ ID NO: 14 (AON QRX136.36):
AAGAAUAUGAAGGUCUUCCUC
SEQ ID NO: 15 (AON QRX136.37):
UGAAGAAUAUGAAGGUCUUCC
SEQ ID NO: 16 (AON QRX136.38):
GUGAUGAAGAAUAUGAAGGUC
SEQ ID NO: 17 (AON QRX136.39):
CUAAUCUGUGAUGAAGAAUAUG
SEQ ID NO: 18 (AON QRX136.40):
AGUUCUAAUCUGUGAUGAAGA
SEQ ID NO: 19 (AON QRX136.41):
CCUGUAGUUCUAAUCUGUGAUG
SEQ ID NO: 20 (AON QRX136.42):
GCCUGUAGUUCUAAUCUGUGA
SEQ ID NO: 21 (AON QRX136.43):
CUAUCAGCCUGUAGUUCUAAUC
SEQ ID NO: 22 (AON QRX136.44):
AGUGAACUAUCAGCCUGUAGU
SEQ ID NO: 23 (AON QRX136.45):
UUUAUUUAGUGAACUAUCAGCC
SEQ ID NO: 24 (AON QRX136.46):
AAUUUAUUUAGUGAACUAUCA
SEQ ID NO: 25 (AON QRX136.47):
CCGUUUGUUUGAAUUUAUUUAG
SEQ ID NO: 26 (AON QRX136.48 and QRX136.48a):
CCAAGCCGUUUGUUUGAAUUUA
SEQ ID NO: 27 (AON QRX136.49 and QRX136.49a):
ACCCAAGCCGUUUGUUUGAAUU
SEQ ID NO: 28 (AON QRX136.50 and QRX136.50a):
CUUACCCAAGCCGUUUGUUUGA
SEQ ID NO: 29 (AON QRX136.51 and QRX136.51a):
CUUAGAAUCUUACCCAAGCCG
SEQ ID NO: 30 (AON QRX136.52):
UGGAACAAAGUUCUUAGAAUC
SEQ ID NO: 31 (ddPCR primer/probe
hCEP290_e34_ctrl_Fw):
CGGGCAACTTGCAAATCACTA
SEQ ID NO :32 (ddPCR primer/probe
hCEP290_e35_ctrl_Hex):
AATCTGCTTTAAGGTTAGCAGAACA
SEQ ID NO: 33 (ddPCR primer/probe
hCEP290_e35_ctrl_Rv):
GGCAATCGAAGCCTCAGTTC
SEQ ID NO: 34 (ddPCR primer/probe
hCEP290_e35_skip_Fw_2):
ACCATTGCAAACATGCAAGC
SEQ ID NO: 35 (ddPCR primer/probe
hCEP290_e35-37_skip_Fam; the 4^{th} and
20^{th} nucleotide are LNA):
TGTTTCATTAAATCCTCTCTGGC
SEQ ID NO: 36 (ddPCR primer/probe
hCEP290_e37_skip_Rv_2):
TGTTCCATCTCAGCCAGACG
SEQ ID NO: 37 (AON QRX136.30a):
CUUCACAAUUUCUCUUUGCUCC
SEQ ID NO: 38 (AON QRX136.33a):
CUUCCUCAUGUUUCUUCACAA
SEQ ID NO: 39 (AON QRX136.34a):
GAAGGUCUUCCUCAUGUUUCUU
SEQ ID NO: 40 (AON QRX136.53a):
CAAUUUCUCUUUGCUCCUGUU
SEQ ID NO: 41 (AON QRX136.54a):
GGUCUUCCUCAUGUUUCUUCA
SEQ ID NO: 42 (Box 1; target sequence):
GGAGCAAAGAG
SEQ ID NO: 43 (complementary sequence
opposite to Box 1):
CUCUUUGCUCC
SEQ ID NO: 44 (Box 2 mutant; target sequence;
first position T =c.4723A>T mutation):
TAGAAACATGAGGAAG
SEQ ID NO: 45 (complementary sequence
opposite to Box 2 mutant):
CUUCCUCAUGUUUCUA
SEQ ID NO: 46 (Box 2 wild type;
target sequence):
AAGAAACATGAGGAAG
SEQ ID NO: 47 (complementary sequence
opposite to Box 2 wild type):
CUUCCUCAUGUUUCUU
SEQ ID NO: 48 (Box 3; target sequence):
TCAAACAAACGGCTTGG
SEQ ID NO: 49 (complementary sequence
opposite to Box 3, present in AON):
CCAAGCCGUUUGUUUGA
SEQ ID NO: 50 (negative control AON):
GUCCCAUCAUUCAGGUCCAUGGCA
SEQ ID NO: 51 (AON QRX136.55a):
AAGGUCUUCCUCAUGUUUCU
SEQ ID NO: 52 (AON QRX136.56a):
AGGUCUUCCUCAUGUUUC
SEQ ID NO: 53 (AON QRX136.57a):
CCAAGCCGUUUGUUUGAAUU
SEQ ID NO: 54 (AON QRX136.58a):
CCAAGCCGUUUGUUUGAA
SEQ ID NO: 55 (AON QRX136.59a):
CCAAGCCGUUUGUUUG
SEQ ID NO: 56 (AON QRX136.60a):
UUACCCAAGCCGUUUGUUUG
SEQ ID NO: 57 (AON QRX136.61a):
CUUACCCAAGCCGUUUGU
SEQ ID NO: 58 (AON QRX136.62a):
UUACCCAAGCCGUUUG
SEQ ID NO: 59 (AON QRX136.63a):
UAGAAUCUUACCCAAGCCG
SEQ ID NO: 60 (AON QRX136.64a):
GAAUCUUACCCAAGCCG
SEQ ID NO: 61 (AON QRX136.65a):
AUCUUACCCAAGCCG
SEQ ID NO: 62 (AON QRX136.66a):
AGGUCUUCCUCAUGUUUCU
SEQ ID NO: 63 (AON QRX136.67a):
GGUCUUCCUCAUGUUUCU
SEQ ID NO: 64 (AON QRX136.68a):
GUCUUCCUCAUGUUUCU
SEQ ID NO: 65 (AON QRX136.69a):
UCUUCCUCAUGUUUCU
SEQ ID NO: 66 (AON QRX136.70a):
GGUCUUCCUCAUGUUUC
SEQ ID NO: 67 (AON QRX136.71a):
GUCUUCCUCAUGUUUC
SEQ ID NO: 68 (AON QRX136.72a):
AGGUCUUCCUCAUGUUU
SEQ ID NO: 69 (AON QRX136.73a):
AGGUCUUCCUCAUGUU
SEQ ID NO: 70 (AON QRX136.74a):
AAGGUCUUCCUCAUGUUU
SEQ ID NO: 71 (AON QRX136.75a):
AAGGUCUUCCUCAUGUU
SEQ ID NO: 72 (AON QRX136.76a):
AAGGUCUUCCUCAUGU
SEQ ID NO: 73 (AON QRX136.77a):
GGUCUUCCUCAUGUUU
SEQ ID NO: 74 (AON QRX136.78a):
CCAAGCCGUUUGUUUGA
SEQ ID NO: 75 (AON QRX136.79a):
UUACCCAAGCCGUUUGUU
SEQ ID NO: 76 (AON QRX136.80a):
UUACCCAAGCCGUUUGU
SEQ ID NO: 77 (AON QRX136.81a_49):
AAUCUUACCCAAGCCGUU
SEQ ID NO: 78 (AON QRX136.81a_50):
AAUCUUACCCAAGCCGU
SEQ ID NO: 79 (AON QRX136.82a):
AGAAUCUUACCCAAGCCG
SEQ ID NO: 80 (AON QRX136.83a):
UUAGAAUCUUACCCAA
SEQ ID NO: 81 (AON QRX136.84a):
UUAGAAUCUUACCCAAG
SEQ ID NO: 82 (AON QRX136.85a):
UUAGAAUCUUACCCAAGC
SEQ ID NO: 83 (AON QRX136.86a):
UAGAAUCUUACCCAAG
SEQ ID NO: 84 (AON QRX136.87a):
AGAAUCUUACCCAAGC
SEQ ID NO: 85 (AON QRX136.88a):
UAGAAUCUUACCCAAGCC
SEQ ID NO: 86 (AON QRX136.89a):
AGAAUCUUACCCAAGCC
SEQ ID NO: 87 (AON QRX136.90a):
GAAUCUUACCCAAGCC
SEQ ID NO: 88 (AON m35ESE and m35ESEa);
CAUGAAGGUCUUCCUCAUGC
SEQ ID NO: 89 (AON h35ESE and h35ESEa):
UAUGAAGGUCUUCCUCAUGU
SEQ ID NO: 90 (AON m35D and m35Da):
GUUCUCAGAAUCUUACCUGAGCUG
SEQ ID NO: 91 (AON h35D and h35Da):
GUUCUUAGAAUCUUACCCAAGCCG
SEQ ID NO: 92 (AON H36D and H36Da):
UAGAAUCUUACCCAAGCCGUUU
SEQ ID NO: 93 (QRX.136.101a):
AAGCCGUUUGUUUGAAUUUA
SEQ ID NO: 94 (QRX.136.102a):
AAGCCGUUUGUUUGAAUUU
SEQ ID NO: 95 (QRX.136.103a):
AAGCCGUUUGUUUGAAUU
SEQ ID NO: 96 (QRX.136.104a):
AAGCCGUUUGUUUGAAU
SEQ ID NO: 97 (QRX.136.105a):
AAGCCGUUUGUUUGAA
SEQ ID NO: 98 (QRX.136.106a):
AAGCCGUUUGUUUGA
SEQ ID NO: 99 (QRX.136.107a):
CAAGCCGUUUGUUUGAAUUU
SEQ ID NO: 100 (QRX.136.108a):
CAAGCCGUUUGUUUGAAUU
SEQ ID NO: 101 (QRX.136.109a):
CAAGCCGUUUGUUUGAAU
SEQ ID NO: 102 (QRX.136.110a):
CAAGCCGUUUGUUUGAA
SEQ ID NO: 103 (QRX.136.111a):
CAAGCCGUUUGUUUGA
SEQ ID NO: 104 (QRX.136.112a):
CAAGCCGUUUGUUUG
SEQ ID NO: 105 (QRX136.113a):
CCAAGCCGUUUGUUUGAAU
SEQ ID NO: 106 (QRX.136.114a):
CCAAGCCGUUUGUUU
SEQ ID NO: 107 (QRX.136.115a):
CCCAAGCCGUUUGUUUGAAU
SEQ ID NO: 108 (QRX.136.116a):
CCCAAGCCGUUUGUUUGAA
SEQ ID NO: 109 (QRX.136.117a):
CCCAAGCCGUUUGUUUGA
SEQ ID NO: 110 (QRX.136.118a):
CCCAAGCCGUUUGUUUG
SEQ ID NO: 111 (QRX.136.119a):
CCCAAGCCGUUUGUUU
SEQ ID NO: 112 (QRX.136.120a):
CCCAAGCCGUUUGUU
SEQ ID NO: 113 (QRX.136.121a):
ACCCAAGCCGUUUGUUUGAA
SEQ ID NO: 114 (QRX.136.122a):
ACCCAAGCCGUUUGUUUGA
SEQ ID NO: 115 (QRX.136.123a):
ACCCAAGCCGUUUGUUUG
SEQ ID NO: 116 (QRX.136.124a):
ACCCAAGCCGUUUGUUU
SEQ ID NO: 117 (QRX.136.125a):
ACCCAAGCCGUUUGUU
SEQ ID NO: 118 (QRX.136.126a):
ACCCAAGCCGUUUGU
SEQ ID NO: 119 (QRX.136.127a):
UACCCAAGCCGUUUGUUUGA
SEQ ID NO: 120 (QRX.136.128a):
UACCCAAGCCGUUUGUUUG
SEQ ID NO: 121 (QRX.136.129a):
UACCCAAGCCGUUUGUUU
SEQ ID NO: 122 (QRX.136.130a):
UACCCAAGCCGUUUGUU
SEQ ID NO: 123 (QRX.136.131a):
UACCCAAGCCGUUUGU
SEQ ID NO: 124 (QRX.136.132a):
UACCCAAGCCGUUUG
SEQ ID NO: 125 (QRX.136.133a):
UUACCCAAGCCGUUUGUUU
SEQ ID NO: 126 (QRX.136.134a):
UUACCCAAGCCGUUU
SEQ ID NO: 127 (QRX.136.135a):
CUUACCCAAGCCGUUUGUUU
SEQ ID NO: 128 (QRX.136.136a):
CUUACCCAAGCCGUUUGUU
SEQ ID NO: 129 (QRX.136.137a):
CUUACCCAAGCCGUUUG
SEQ ID NO: 130 (QRX.136.138a):
CUUACCCAAGCCGUUU
SEQ ID NO: 131 (QRX.136.139a):
CUUACCCAAGCCGUU
SEQ ID NO: 132 (QRX.136.140a):
UCUUACCCAAGCCGUUUGUU
SEQ ID NO: 133 (QRX.136.141a):
UCUUACCCAAGCCGUUUGU
SEQ ID NO: 134 (QRX.136.142a):
UCUUACCCAAGCCGUUUG
SEQ ID NO: 135 (QRX.136.143a):
UCUUACCCAAGCCGUUU
SEQ ID NO: 136 (QRX.136.144a):
UCUUACCCAAGCCGUU
SEQ ID NO: 137 (QRX.136.145a):
UCUUACCCAAGCCGU
SEQ ID NO: 138 (QRX.136.146a):
AUCUUACCCAAGCCGUUUGU
SEQ ID NO: 139 (QRX.136.147a):
AUCUUACCCAAGCCGUUUG
SEQ ID NO: 140 (QRX.136.148a):
AUCUUACCCAAGCCGUUU
SEQ ID NO: 141 (QRX.136.149a):
AUCUUACCCAAGCCGUU
SEQ ID NO: 142 (QRX.136.150a):
AUCUUACCCAAGCCGU
SEQ ID NO: 143 (QRX.136.151a):
AAUCUUACCCAAGCCGUUUG
SEQ ID NO: 144 (QRX.136.152a):
AAUCUUACCCAAGCCGUUU
SEQ ID NO: 145 (QRX.136.153a):
AAUCUUACCCAAGCCG
SEQ ID NO: 146 (QRX.136.154a):
AAUCUUACCCAAGCC
SEQ ID NO: 147 (target sequence):
TAAATAAATTCAAACAAACGGCTTGGgtaagatt
SEQ ID NO: 148 (control AON):
CUUAAAGAUGAUCUCUUACC
SEQ ID NO: 149 (control AON USH2a-PE40-43):
AGAUUCGCUGCUCUUGUU

### EXAMPLES

### Example 1: Design of Antisense Oligonucleotides to Induce the Skipping of Exon 36 from the Human CEP290 Pre-mRNA

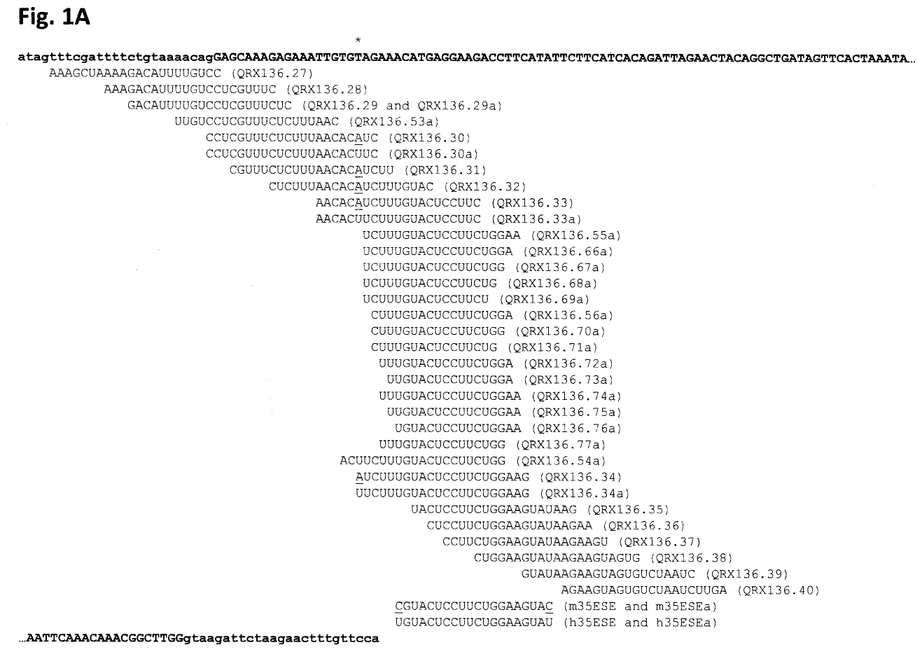

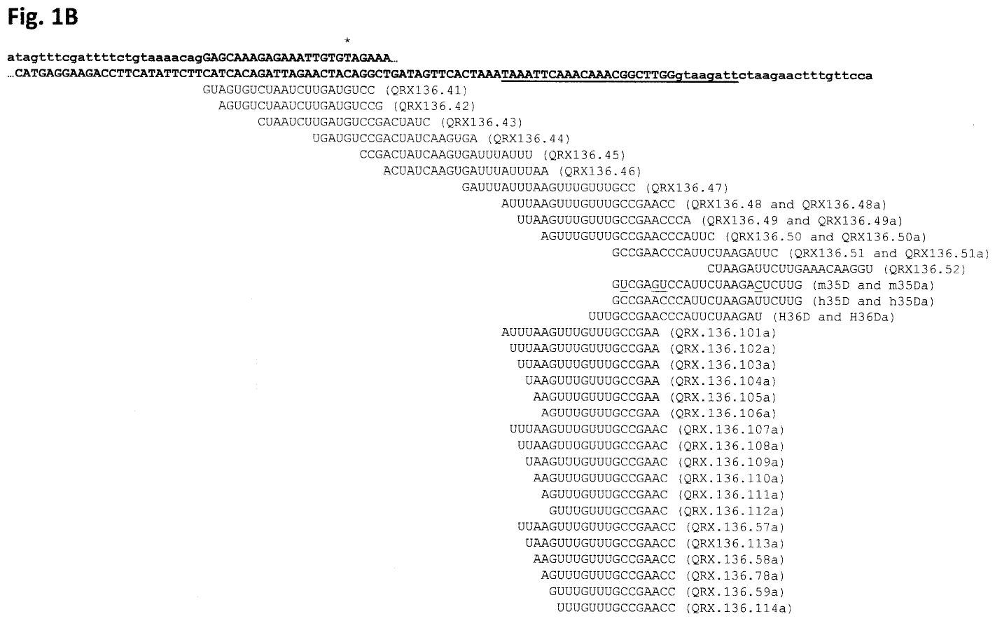

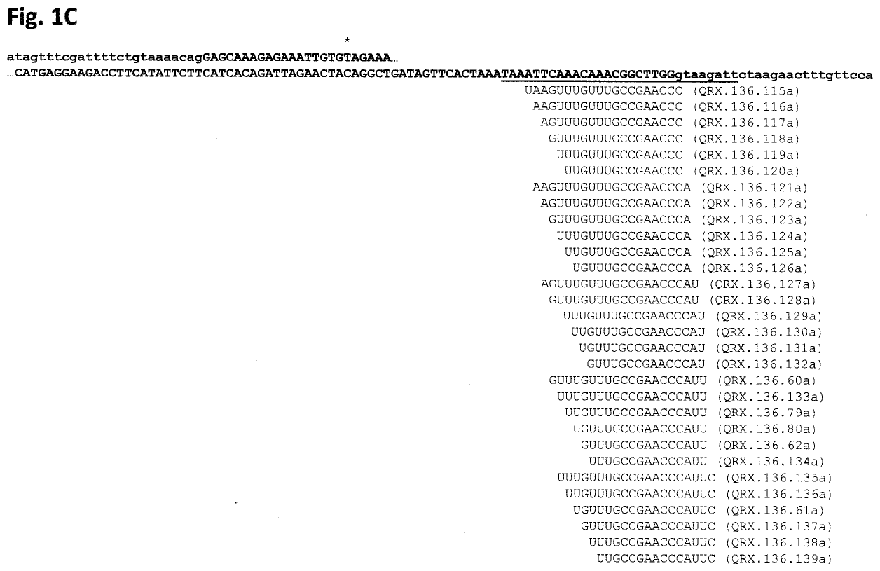

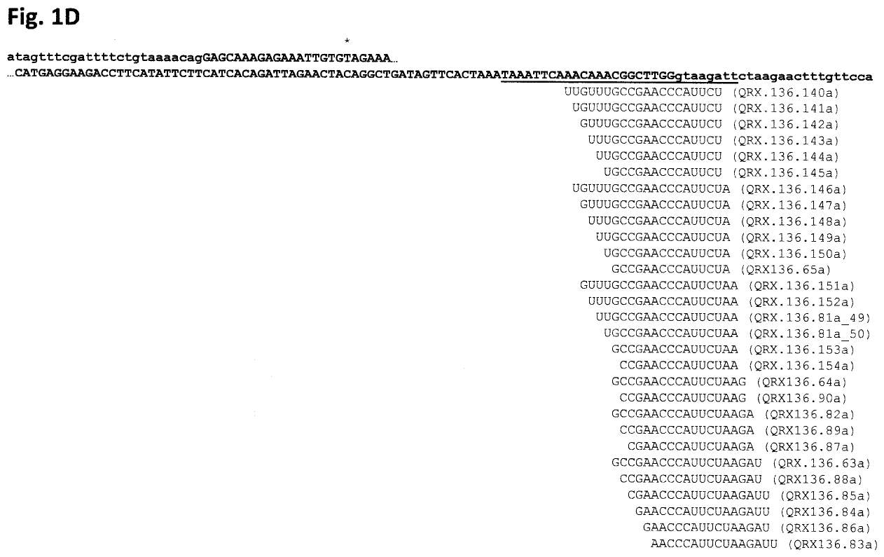

Initially twenty-six antisense oligonucleotides (AONs QRX136.27 to QRX136.52; full RNA) were designed using a genome-walking approach in which all sequences of exon 36, as well as part of the upstream intron 35 and part of the downstream intron 36 were covered. AONs QRX136.30, -31, -32, -33 and -34 were generated with an adenosine (A) opposite the position of the c.4723A>T mutation (mutation given by an asterisk (*) and A nucleotides in the respective AONs given underlined in FIG. 1). All twenty-six AONs (without an “a” in the name) were modified with a 2′-O-methyl (2′-OMe) modification on each of their sugar moieties. QRX136.53a and QRX136.54a were subsequently designed and carry both a 2′-MOE substitution. QRX136.29a, QRX136.48a, QRX136.49a, QRX136.50a and QRX136.51a have an identical sequence as their counterparts, but instead are fully 2′-MOE modified. QRX136.30a, QRX136.33a and QRX136.34a are also fully 2′-MOE modified, but carry a uridine (U) instead of an adenosine (A) opposite the position of the c.4723A>T mutation, to be 100% complementary to the wild type sequences (when tested in wild type human fibroblasts, see below). All internucleoside linkages in each of the AONs were modified with phosphorothioate. All AONs, when prepared, were resolved in RNase/DNase free water to a concentration of 100 μM.

Additional AONs were designed to be either shorter or longer at either end of the best performing oligonucleotides, and generally carry a full 2′-MOE modified backbone. These AONs, when targeting a region covering the mutation site carry a uridine (U) when tested in wild type cells or tissue. Equivalent AONs contain an adenosine (A) opposite the position of the c.4723A>T mutation when assayed in LCA10 patient material (generally fibroblasts derived from an LCA10 patient carrying the c.4723A>T mutation).

### Example 2: CEP290 Exon 36 Skipping by Antisense Oligonucleotides Assayed by Droplet Digital PCR

The twenty-six AONs QRX136.27 to QRX136.52, see FIG. 1, with their respective sequences SEQ ID NO:5 to 30) were used in a transfection assay using non-patient (wild type) fibroblasts. Subsequently, the percentage of skipping was determined using a droplet digital PCR (ddPCR) procedure, as follows.

### Cell Culture

The cells used for transfections were wild type (non-patient) fibroblasts. Cells were cultured at 37° C. and 5% CO₂ in Dulbecco's minimal essential medium (DMEM; Gibco) with 10% fetal bovine serum (FBS; Biowest) and 1% penicillin/streptomycin (P/S; Gibco). Cells were seeded in a 6-well tissue culture plate (Greiner) prior to transfection. After reaching 80% confluency the cells were ready for transfection.

### Transfection

The twenty-six AONs were tested at a concentration of 100 nM. To verify the AON concentration the resolved compounds were first analyzed on the NanoDrop spectrophotometer (NanoDrop 2000; Thermo Scientific). The sample type was set to DNA-50 and prior to the first sample measurement, the spectrophotometer was set to a zero-baseline using RNase/DNase free water as a blank solution.

The human fibroblasts were transfected with 1:4 (μg oligo:μL reagent) MaxPEI (Polysciences Inc.) as suggested by the manufacturer. The transfection mixture was prepared as follows: AON and MaxPEI were added to 100 μL NaCl (150 mM), mixed vigorously and incubated at RT for 15 min. Consecutively, the culture medium was aspirated and replaced with 900 μL transfection medium, i.e. culture medium lacking P/S. After incubation the transfection mixture was added into the transfection medium and mixed gently. Immediately thereafter the plates were placed back in the incubator at 37° C. for continued growth. 4 h after starting the transfection the entire volume was aspirated and replaced with 1 mL regular culture medium. Thereafter the cells were cultured as normal for another 20 h. All AONs were tested in duplicate.

### RNA Extraction

For RNA extraction the RNeasy Plus Mini Kit (Qiagen) was used according the manufacturer's protocol. Samples were first lysed on the plate in 350 μL lysis buffer (+10 μL/mL (3-mercaptoethanol) and then transferred to a new tube and centrifuged at max speed for 3 min. The supernatant (lysate) was passed through a gDNA Eliminator spin column, ethanol was added to the flow-through, and the sample was applied to an RNeasy MinElute spin column. Contaminants were washed away in three wash steps. Finally, the RNA was eluted in 30 μL of RNase free water. Extracted RNA was stored at −20° C.

### Reverse Transcription

Per sample, 500 ng RNA was reverse transcribed using the Maxima H Minus First Strand cDNA Synthesis Kit, with dsDNase (Thermo Fisher Scientific). Prior to cDNA synthesis the RNA concentration was determined using the NanoDrop spectrophotometer at RNA-40 and using the extraction elution solution as a blank. cDNA was prepared using random hexamers, dNTPs and the RT enzyme supplied in the kit, in a 20 μL reaction according to the manufacturer's protocol.

For each sample, the following mixture was prepared: 13 μL sample (500 ng RNA made up to 13 μL with RNase free water)+4 μL 5× RT Buffer+1 μL hexamers (400 ng/μL)+1 μL dNTPs (10 mM)+1 μL Maxima H Minus Enzyme Mix. Two negative controls were included: 1) RT(−), consisting of a second mix of the sample with the highest concentration and water instead of RT enzyme; and 2) NTC, using water instead of RNA in combination with all other reagents. Cycle conditions: a cDNA synthesis step for 30 min at 50° C., reaction termination for 5 min at 85° C., followed by an unlimited holding step at 4° C. Resulting cDNA was stored at −20° C.

Droplet Digital PCR (ddPCR)

Equipment and reagents were obtained from Bio-Rad. All samples were analyzed in duplicate. Control and skip detection assays were analyzed separately, as given below. For all reactions, a mixture was prepared containing per sample: 2 μL undiluted cDNA template, 2×ddPCR™ Supermix for Probes (no dUTP) and primers/probes at a final concentration of 250/160 nM respectively in a total reaction volume of 20 μL. The following primers/probes were used:

| | | Sequence | |
|---|---|---|---|
| Primer/ | | (5′ to 3′) | |
| probe name | # | (SEQ ID NO) | purpose |
| hCEP290_ | 1 | CGGGCAACTTG | control |
| e34_ | | CAAATCACTA | |
| ctrl_ | | (31) | |
| Fw | | | |
| hCEP290_ | 2 | AATCTGCTTTA | control |
| e35_ | | AGGTTAGCAGA | |
| ctrl_ | | ACA | |
| Hex | | (32) | |
| hCEP290_ | 3 | GGCAATCGAAG | control |
| e35_ | | CCTCAGTTC | |
| ctrl_ | | (33) | |
| Rv | | | |
| hCEP290_ | 19 | ACCATTGCAAA | skip |
| e35_ | | CATGCAAGC | detection |
| skip_ | | (34) | |
| Fw_ | | | |
| 2 | | | |
| hCEP290_ | 20 | TGTTTCATTAA | skip |
| e35-37_ | | ATCCTCTCTGG | detection |
| skip_ | | C | |
| Fam | | (35) | |
| hCEP290_ | 21 | TGTTCCATCT | skip |
| e37_ | | CAGCCAGACG | detection |
| skip_ | | (36) | |
| Rv_ | | | |
| 2 | | | |

The 4^{th} and 20^{th} nucleotide in hCEP290_e35-37_skip_Fam was a Locked Nucleic Acid (LNA), for binding affinity purposes.

The complete assay mix was transferred to sample wells of an eight-channel disposable droplet generator cartridge. After sample loading, the eight oil wells were filed with 70 μL Droplet Generation Oil for Probes and placed in the droplet generator. The generator applies a vacuum to the outlet wells pulling individual samples and oil through a flow-focusing junction to simultaneously partition each 20 μL sample into ˜20,000 0.85 nL sized droplets.

Upon generation completion, the cartridge was removed from the generator and the droplet emulsions (˜40 μL), collected in the independent outlet-wells, were transferred to a 96-well plate. The plate was sealed with a pierce-able foil heat seal using a PX1 PCR Plate Sealer and cycled in a C1000 thermocycler. Thermal cycling conditions for all probe assays were: enzyme activation for 10 min at 95° C., followed by 40 cycles of a two-step thermal profile consisting of a denaturation step (30 sec at 94° C.) and a combined annealing/extension step (60 sec at 60° C.). An enzyme deactivation step (98° C. for 10 min) was performed at the end of the thermal cycling protocol before finally maintaining the temperature at 4° C. until further use.

After PCR, the 96-well plate was loaded into the QX200 Droplet Reader and the appropriate assay information was entered into QuantaSoft, the accompanying ddPCR analysis software package. Droplets were automatically aspirated from each well and streamed single-file past a two-color fluorescence detector. The quality of all droplets was analyzed, and outliers were gated based on detector peak width. Droplets were assigned as positive or negative based on their fluorescence amplitude. The number of positive and negative droplets in each channel was used to calculate the concentration of the target DNA sequences (copies/μL) and their Poisson-based 95% confidence intervals.

### Data Analysis

The end values resulting from the ddPCR measurement were analyzed as follows. The primary analysis was performed using the QuantaSoft analysis software. A sample was included in the analysis when the total amount of droplets per well was above 10,000. Additionally, negative control samples were checked for any amplification. The accepted samples were checked for both skipped and wild-type CEP290 positive droplets represented by a blue (FAM) or green (HEX) color, respectively. Gating based on cloud formation and fluorescence amplitude was done automatically if possible and confirmed manually. After gating, the positive droplet count in copies/μL for the two replicates was transported to an Excel file for secondary analysis. First the copy numbers for the two technical duplicates of each samples were averaged. Next the percentage skip was calculated by dividing the copies/μL found with the skip assay by those detected with the control assay. Finally, the percentage skip per AON was calculated by averaging the two biological replicates. The standard error of the mean (SEM) was derived from these final values.

### Results of the 1^{st} Screen

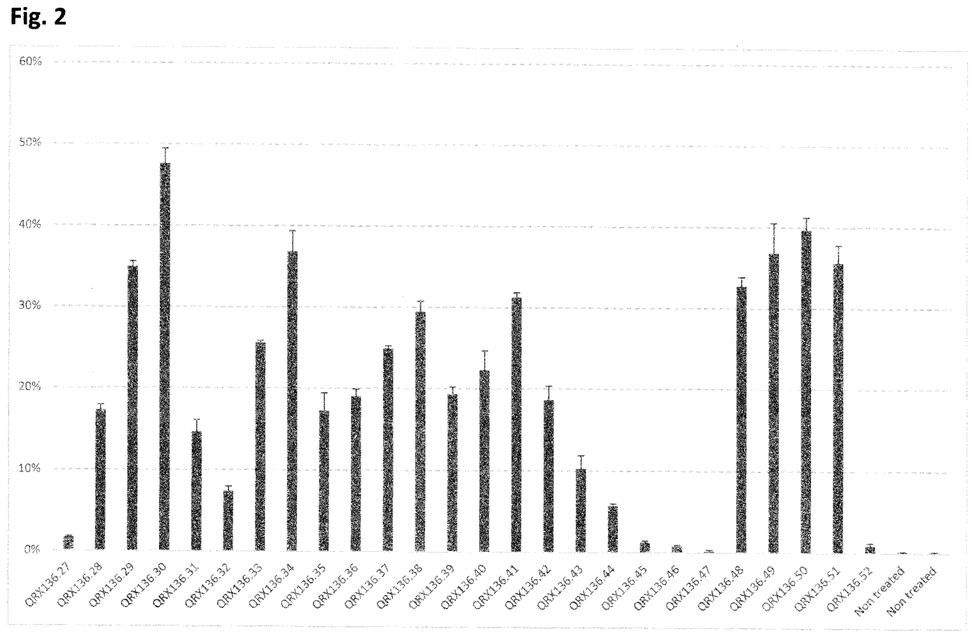

The final percentages of exon 36 skip from the human wild type CEP290 pre-mRNA and the SEM are depicted in FIG. 2. This shows that the background skip (untreated) was close to zero. Except for one AON (QRX136.47 that showed 0.2% skip, similar to the non-treated controls) all tested oligonucleotides showed a certain percentage of skip, with the following approximate percentages:

0 to 2%

### QRX136.27, QRX136.45, QRX136.46, QRX136.47 and QRX136.52

2 to 10%

### QRX136.32 and QRX136.44

10 to 20%

### QRX136.28, QRX136.31, QRX136.35, QRX136.36, QRX136.39, QRX136.42 and QRX136.43

20 to 30%

### QRX136.33, QRX136.37, QRX136.38 and QRX136.40

>30%

### QRX136.29, QRX136.30, QRX136.34, QRX136.41, QRX136.48, QRX136.49, QRX136.50 and QRX136.51

Based on these experiments it was concluded that the inventors were able to achieve exon 36 skipping from human CEP290 pre-mRNA using a variety of AONs spanning the exon 36 sequences and its surrounding intronic sequences, and that the percentage of exon 36 skipping in these wild type human fibroblasts varied. It should be noted that QRX136.30, being the AON giving the highest percentage of skip, contains an adenosine (A) opposite the position of the c.4723A>T mutation, whereas in these wild type fibroblasts that position is in fact an A, not a T. This suggests that the percentage of skip can even be increased using patient-derived fibroblasts, or optic cups (retinal organoids) grown from such patient-derived material, using this particular oligonucleotide, or oligonucleotides that are derived from this sequence, and that may be shifted to the 5′ and/or 3′ end, and that may be somewhat shorter and/or somewhat longer than QRX136.30. Notably, whereas QRX136.47 and QRX136.52 resulted in exon 36 skipping that hardly was over background, the AONs located in between these two AONs: QRX136.48, -49, -50, and -51 gave very high exon 36 skip percentages, indicating a hot spot for exon 36 targeting to achieve exon skipping. Known AON m35D (and its ‘human’ equivalent AON h35D) targets a region that is further towards the 3′ end of the transcript, and only partly covers this area (see FIG. 1). As can be seen in FIG. 2, transfections with AONs that target regions in the 5′ part of exon 36 also resulted in high exon 36 skipping percentages.

It was concluded that for the first time it was shown that exon 36 can be skipped from human CEP290 pre-mRNA. This indicates that a method using oligonucleotides for the treatment of LCA10 that is caused by mutations, such as the c.4723A>T mutation in exon 36, is a feasible approach.

### Example 3: Additional AONs for Skipping of CEP290 Exon 36

In a similar set up as described above, ten subsequent AONs were tested for their efficiency to induce the skip of exon 36 from human CEP290 pre-mRNA. Since the previous examples had shown that several regions could be identified as ‘hot spots’ as far as AON targeting goes, the inventors tested the new AONs in a similar experiment as described in example 2:

QRX136.29a

QRX136.53a

QRX136.30a

QRX136.33a

QRX136.54a

QRX136.34a

QRX136.48a

QRX136.49a

QRX136.50a

QRX136.51a

The positions of these AONs is shown in FIG. 1. In these names, the “a” represents the fact that in contrast to the AONs tested in example 2, these ten additional AONs were fully 2′-MOE modified. It is noted that QRX136.30a, QRX136.33a and QRX136.34a have a different sequence than their respective counterparts used in example 2, namely that these are 100% complementary to (a part of) the wild type sequence (SEQ ID NO:2). This enabled the inventors to test their exon 36 skipping capabilities using wild type (non-patient) fibroblasts.

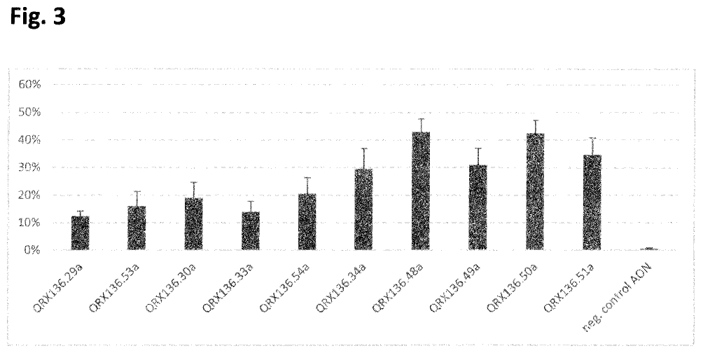

The additional ten 2′-MOE modified AONs were transfected in wild type human fibroblasts using a concentration of 50 nM each using Lipofectamine 2000 (Thermo Fisher scientific) at a ratio of 1:5 (μg oligo:μL reagent). A non-CEP290 targeting 2′-MOE modified AON of similar length (5′-GUCCCAUCAUUCAGGUCCAUGGCA-3′; SEQ ID NO:50) was used as a negative control. Otherwise the experiment was performed as described above, using four transfections, and in duplicate for each AON. The averaged ddPCR results after using these ten additional AONs are depicted in FIG. 3 and show that all these AONs performed well in exon 36 skipping, with again QRX136.48a, -49a, -50a and -51a outperforming the other AONs, similar to what was found in the previous example. Interestingly QRX136.30a did not perform as efficiently as its QRX136.30 counterpart, perhaps also because their sequences were not 100% identical (see above). Results in percentages skip were as follows:

10 to 20%

### QRX136.29a, QRX136.53a, QRX136.30a, QRX136.33a

20 to 30%

### QRX136.54a, QRX136.34a

>30%

### QRX136.48a, QRX136.49a, QRX136.50a, QRX136.51a

Notably, half the amount of AON was used in these experiments as compared to the transfection performed in the previous example, while percentages of skip appeared comparable, These results suggest that the skipping efficiency of an AON not only depends on the sequence and the target region, but that different 2′ substitutions may add in their respective skipping efficiencies.

### Example 4: Testing Additional 2′-MOE Modified AONs for their Efficiency to Skip Mutant Exon 36 from CEP290 Pre-mRNA after Transfection Versus Gymnotic Uptake

Based on the results outlined in the previous examples, it was concluded that the two regions previously identified: 1) the first being the region surrounding the ESE in the 5′ part of exon 36 and 2) the second being the region towards the 3′ terminal part of exon 36 and partly overlapping the 5′ sequence of the downstream intron, appeared to be the major hotspots for targeting exon 36 to modulate its splicing and exclusion from the human CEP290 mRNA. It appeared that the AONs targeting the 3′ terminus of exon 36 were particularly preferred, especially the region covered by the QRX136.48(a), -49(a), -50(a) and -51(a) AONs.

In a further experiment, yet more AONs were tested that were fully modified with 2′-MOE substitutions and that especially targeted the 3′ terminus of exon 36, with some AONs overlapping with the 5′ terminus of the downstream intron 36. This was particularly done to determine whether shorter versions of the earlier tested AONs would provide improved results. The three 22-nucleotide long AONs tested in the examples above, QRX136.34a, -48a, and -50a where compared both in transfection and gymnotic uptake experiments with the following eleven additional AONs (with their length given between brackets):

QRX136.55a (20-mer)

QRX136.56a (18-mer)

QRX136.57a (20-mer)

QRX136.58a (18-mer)

QRX136.59a (16-mer)

QRX136.60a (20-mer)

QRX136.61a (18-mer)

QRX136.62a (16-mer)

QRX136.63a (19-mer)

QRX136.64a (17-mer)

QRX136.65a (15-mer)

The cells used for these treatments were human retinoblastoma WERI-Rb-1 cells. Cells were cultured at 37° C. and 5% CO₂ in Roswell Park Memorial Institute 1640 medium (RPMI 1640; Gibco) with 10% fetal bovine serum (FBS; Biowest). 500.000 cells were seeded per well in a 12-well tissue culture plate (Greiner) prior to treatment. Immediately after seeding the cells were subjected to 48 hrs of AON treatment. 100 nM AON was used with either Lipofectamine 2000 (Thermo) transfection reagent for transfections. 10 μM AON was used without transfection reagents (generally referred to as ‘gymnotic uptake’). Subsequently percentages of exon 36 skip were determined as described above. A 20-mer 2′-MOE control AON was taken along (5′-CUUAAAGAUGAUCUCUUACC-3′; SEQ ID NO:148).

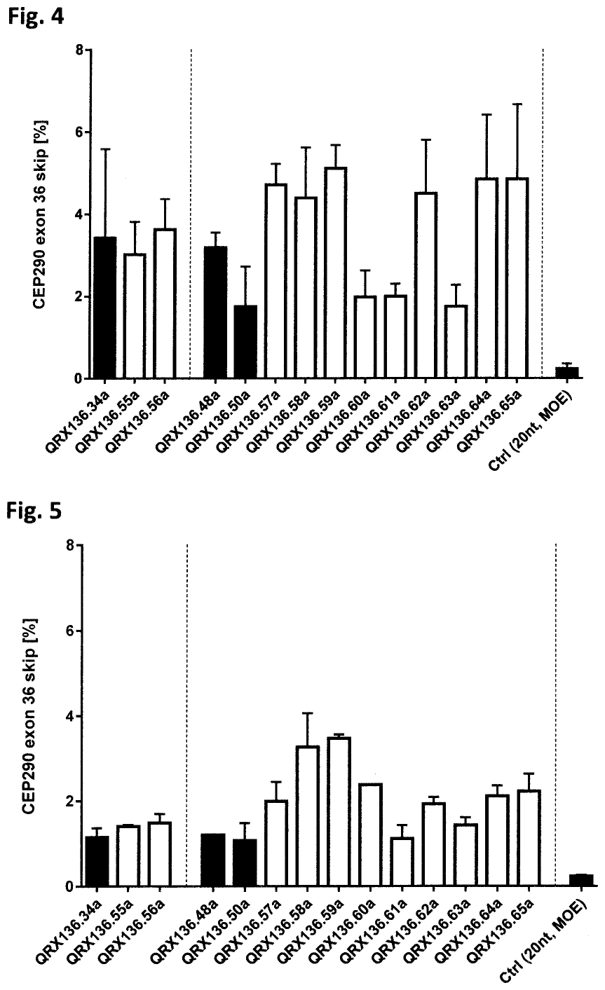

Results of the transfection experiment are shown in FIG. 4, whereas the results of the gymnotic uptake experiment are shown in FIG. 5. This shows that shorter versions of the AONs targeting the 5′ part of exon 36 (QRX136.55a and QRX136.56a) in comparison to a longer version (QRX136.34a) did not give a significantly improved result. However, in the region close to the splice donor site, the short AONs (QRX136.57a, QRX136.58a, QRX136.62a, QRX136.64a and QRX136.65a) significantly outperformed the longer versions (QRX136.48a and QRX136.50a) under gymnotic uptake conditions. This indicates that exon skipping was increased at the 3′ end of exon 36 using the shorter AONs in a gymnotic uptake environment.

### Example 5: Comparing 2′-MOE Modified AONs Under Gymnotic Uptake Conditions for Ability to Provide Exon 36 Skip from Human CEP290 mRNA

Based on the results described above, an additional set of oligonucleotides was tested to detect the best targeting area for yielding exon 36 skipping and to determine how the newly identified AONs would compare to the AONs known from WO 2015/004133, Gerard et al. (2015) and Barny et al. (2019), when these were transformed to a human-CEP290 specific version. These AONs were also manufactured such that all nucleosides were modified to carry a 2′-MOE substitution (h35ESEa, h35 Da and H36 Da). The following AONs were tested in a similar gymnotic uptake experiment in human WERE-Rb-1 cells as outlined above and percentages of exon 36 skip were determined by ddPCR as outlined above, listed by their respective target areas:

| | |
|---|---|
| QRX136.66a | QRX136.58a |
| QRX136.67a | QRX136.78a |
| QRX136.68a | QRX136.59a |
| QRX136.69a | QRX136.79a |
| QRX136.56a | QRX136.80a |
| QRX136.70a | QRX136.81a_49 |
| QRX136.71a | QRX136.81a_50 |
| QRX136.72a | QRX136.64a |
| QRX136.73a | QRX136.83a |
| QRX136.74a | QRX136.84a |
| QRX136.75a | QRX136.85a |
| QRX136.76a | QRX136.86a |
| QRX136.77a | QRX136.87a |
| m35ESE | QRX136.88a |
| h35ESE | QRX136.89a |
| m35ESEa | QRX136.90a |
| h35ESEa | m35D |
| | h35D |
| | m35Da |
| | h35Da |
| | H36D |
| | H36Da |

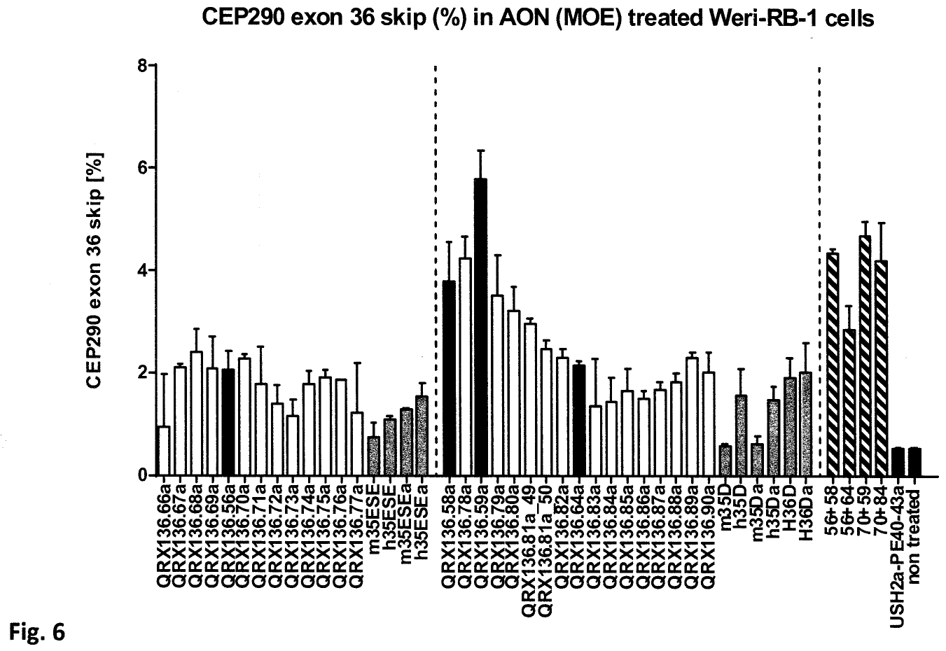

The results obtained with the AONs targeting the more 5′ exon 36 area are shown in FIG. 6 on the left part of the diagram. It appeared that QRX136.67a (18-mer), QRX136.68a (17-mer), QRX136.69a (16-mer), QRX136.56a (18-mer), QRX136.70a (17-mer), QRX136.71a (16-mer), QRX136.74a (18-mer), QRX136.75a (17-mer), and QRX136.76a (16-mer) outperformed the best performing AON derived from the art: 20-mer h35ESEa, which is the ‘humanized’ and 2′-MOE modified version of m35ESE.

In respect of the AONs targeting the region at the 3′ terminus of exon 36 and partly overlapping with the 5′ terminus of downstream intron 36 (FIG. 6, middle part), these results were even more striking: gymnotic uptake of AONs QRX136.58a (18-mer; SEQ ID NO:54), QRX136.78a (17-mer; SEQ ID NO:74), QRX136.59a (16-mer; SEQ ID NO:55), QRX136.79a (18-mer; SEQ ID NO:75), QRX136.80a (18-mer; SEQ ID NO:76), QRX136.81_49 (18-mer; SEQ ID NO:77), and QRX136.81_50 (17-mer; SEQ ID NO:78) resulted in a significantly higher exon 36 skipping percentage than what could be detected after gymnotic uptake of the best performing AON from the art (22-mer H36 Da, which is a 2′-MOE modified version of H36D (SEQ ID NO:92)). Furthermore, the results from this experiment, together with the results described above allowed the inventors to identify the hotspot area that can most beneficially be targeted with a fully complementary 15-, 16-, 17-, 18-, 19-, and/or 20-nucleotide AON for most sufficient exon 36 skipping from human CEP290. This hotspot target area is depicted as a 30-nucleotide target region in FIG. 1 (underlined in the bold sequence) and which is provided herein as SEQ ID NO:147.

Besides the experiments with the new set of AONs, the inventors also combined several AONs, using one AON targeting the Box 2 region, and one targeting the Box 3 region, using the following combinations:

a) QRX136.56a+QRX136.58a

b) QRX136.56a+QRX136.64a

c) QRX136.70a+QRX136.59a

d) QRX136.70a+QRX136.84a

The AONs in the combination experiment were mixed 50/50, together adding up to 10 μM AON (5 μM each), equal to the concentration of AON in the experiments using a single AON. As a control an 18-mer 2′-MOE oligonucleotide was taken along (USH2a-PE40-43; 5′-AGAUUCGCUGCUCUUGUU-3′; SEQ ID NO:149) together with a sample not treated with AONs. The results are depicted on the right part of FIG. 6. While it may be concluded that the relative high exon 36 skip percentage found with combination a) and c) was due to the presence of QRX136.58a and QRX136.59a, respectively (although present in half the percentage as compared to the gymnotic uptake using a single AON), it was surprisingly found that combinations b) and d) provided a higher exon 36 skip percentage than found when respective AONs were used alone.

The identified hotspot target region of 30 nucleotides (SEQ ID NO:147), when targeted with a 15-, 16-, 17-, 18-, 19-, or 20-mer AON allows for a range of 66 oligonucleotides in total (see FIG. 1). To further substantiate the feasibility of using any 100% complementary AON of this length (15, 16, 17, 18, 19, or 20 nucleotides) targeting a consecutive strand of nucleotides of the same length anywhere within this region, the additional AONs (on top of those that served as an example and outlined above) are provided with their respective sequences herein as QRX136.101a to QRX136.154a (SEQ ID NO:93 to 146) and are tested for their efficiency in exon 36 skipping from human CEP290 (pre-) mRNA using generally the same methodology as outlined above.

### Example 6: Testing AONs for Exon 36 Skip from Human CEP290 mRNA in Optic Cups

Wild-type induced pluripotent stem cells (iPSC) were differentiated into retinal organoids (also referred to as ‘optic cups’) and cultured for approximately 180 days using a differentiation protocol based on the methods as described by Hallam et al. (2018. Human-induced pluripotent stem cells generate light responsive retinal organoids with variable and nutrient-dependent efficiency. Stem Cells 36(10):1535-1551) and Kuwahara et al. (2015. Generation of a ciliary margin-like stem cell niche from self-organizing human retinal tissue. Nat Commun 6:6286). After differentiation, organoids were separately treated with 1.5 or 6 μM (for first 10 days), followed by respective concentrations of 3 and 9 μM AON (from day 11 to 28) using AONs QR136.34a, QRX136.48a, and QRX136.61a. As a control, organoids were treated with 6 μM control non-targeting AON for 28 days. Every 2 days, half of the culture medium was refreshed with fresh culture medium containing AONs. After 28 days, organoids were collected, and RNA was extracted using the Direct-zol RNA Microprep kit (Zymo Research) following the recommendations of the manufacturer. cDNA was synthesized with 250 ng RNA using the Verso cDNA synthesis kit (Thermo Fisher Scientific) using the recommendations of the manufacturer. A master mix was prepared containing 4 μL of 5×cDNA synthesis buffer, 2 μL of dNTP mix, 1 μL of RT enhancer, 1 μL of random hexamer primers and 1 μL of Verso enzyme mix per sample. Reactions were incubated at 42° C. for 30 min and heat-inactivated at 95° C. for 2 min. For mRNA quantification of exon 36 skip in human CEP290 mRNA in organoids, ddPCR was performed as described above, using 5 ng of cDNA. In addition, levels of the retinal markers CRX (Hs00230899_m1, Thermo Fisher Scientific), NRL (Hs00172997_m1, Thermo Fisher Scientific) and NR2E3 (Hs00183915_m1, Thermo Fisher Scientific) were measured in the organoids to show that they were well-differentiated. It was confirmed that the organoids were well differentiated (high retinal marker expression and presence of photoreceptors; data not shown).

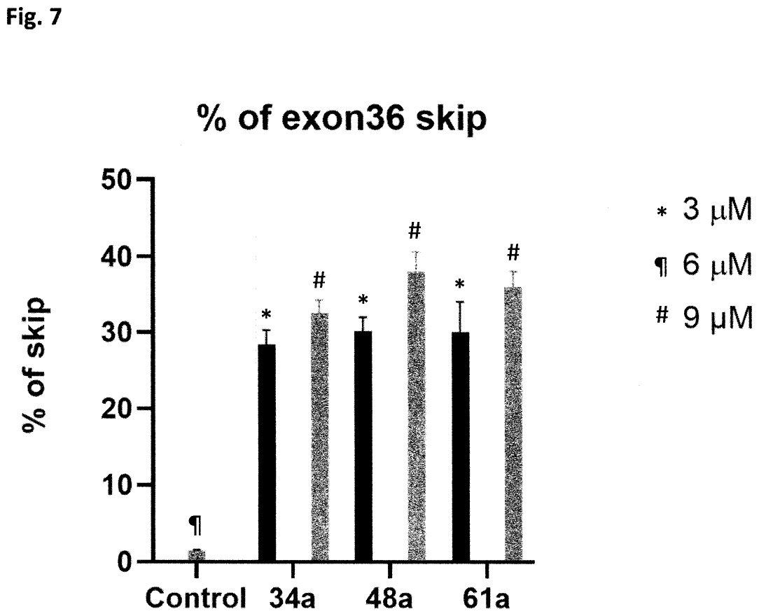

The results (percentages of exon 36 skip) are shown in FIG. 7 and indicate that exon 36 skip from wild-type human CEP290 pre-mRNA could be achieved in these retinal organoids, with percentages between 28% and 36% for all three tested AONs, whereas the control AON gave a skip percentage that was close to zero.

These results show that exon 36 skipping could be achieved in human retinal organoids, again indicating that treatment of LCA10 in humans using any of the preferred AONs as disclosed in the present invention is feasible.

1. An antisense oligonucleotide (AON) capable of inducing skipping exon 36 from human CEP290 pre-mRNA, wherein the AON comprises a sequence that is substantially complementary to a sequence of exon 36 of the human CEP290 gene or a part thereof, or wherein the AON comprises a sequence that is substantially complementary to a sequence of exon 36 of the human CEP290 gene or a part thereof and overlaps with the exon 36/intron 36 boundary at the 3′ end of exon 36 and the 5′ end of intron 36.

2. The AON according to claim 1, wherein the AON comprises or consists of a sequence that is at least 90% complementary to a sequence selected from the group consisting of: SEQ ID NO:42, 44, 46, 48, and 147.

3. The AON according to claim 1 wherein the AON consists of 15, 16, 17, 18, 19, or 20 nucleotides that are 100% complementary to a consecutive sequence within SEQ ID NO:147.

4. The AON according to claim 1, wherein the AON consists of a sequence selected from the group consisting of: SEQ ID NO:7, 8, 11, 12, 15, 16, 18, 19, 26, 27, 28, 29, 37, 38, 39, 40, 41, 51, 52, 53, 54, 55, 56, 57, 58, 59, 60, 61, 63, 64, 65, 66, 67, 70, 71, 72, 74, 75, 76, 77, 78, and 93 to 146.

5. The AON according to claim 1, wherein the AON consists of a sequence selected from the group consisting of: SEQ ID NO:53, 54, 55, 56, 57, 58, 61, 74, 75, 76, 77, 78, and 93 to 146.

6. The AON according to claim 1, wherein the AON consists of a sequence selected from the group consisting of: SEQ ID NO:53, 54, 55, 56, 58, 74, 75, 76, 77, 78, 105, 106, 125, 126, 143, 144, 145, and 146.

7. The AON according to claim 1, wherein the AON comprises at least one non-naturally occurring chemical modification.

8. The AON according to claim 7, wherein the modification comprises at least one non-naturally occurring internucleoside linkage.

9. The AON according to claim 1, wherein the AON comprises one or more sugar moieties that is mono- or di-substituted at the 2′, 3′ and/or 5′ position, wherein the substitution is selected from the group consisting of: —OH; —F; substituted or unsubstituted, linear or branched lower (C1-C10) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

10. The AON according to claim 9, wherein the AON comprises at least one sugar moiety carrying a 2′-OMe modification or a 2′-MOE modification.

11. A pharmaceutical composition comprising an AON according to claim 1, and a pharmaceutically acceptable carrier.

12. The pharmaceutical composition according to claim 11, wherein the pharmaceutical composition is for intravitreal administration and is dosed in an amount ranging from 0.01 mg and 1 mg of total AON per eye.

13. The pharmaceutical composition according to claim 11, wherein the pharmaceutical composition is for intravitreal administration and is dosed in an amount ranging from 0.1 and 1 mg of total AON per eye.

14. A viral vector expressing an AON according to claim 1.

15.-18. (canceled)

19. A method for treating or delaying the onset of Leber's Congenital Amaurosis type 10 (LCA10) comprising administering the AON of claim 1 to a patient in need thereof.

20. A method for treating or delaying the onset of Leber's Congenital Amaurosis type 10 (LCA10) comprising administering the vector of claim 14 to a patient in need thereof.

21. A method for modulating splicing of CEP290 pre-mRNA in a cell, the method comprising contacting the cell with an AON according to claim 1.

22. A method for modulating splicing of CEP290 pre-mRNA in a cell, the method comprising contacting the cell with vector according to claim 14.

23. A method for the treatment of a CEP290-related disease or condition requiring modulating splicing of CEP290 pre-mRNA of an individual in need thereof, the method comprising administering an AON according to claim 1 to a patient in need thereof.

24. A method for the treatment of a CEP290-related disease or condition requiring modulating splicing of CEP290 pre-mRNA of an individual in need thereof, the method comprising administering a vector according to claim 14 to a patient in need thereof.

25. The pharmaceutical composition of claim 13, wherein the composition is dosed at 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 μg total AON per eye.

26. The AON according to claim 9, wherein all nucleosides within the AON are 2-OMe modified or wherein all nucleosides 2′-MOE modified.


SEQUENCE LISTING (149 sequences)

SEQ ID NO: 1 - DNA, 158 bp, Homo sapiens
atagtttcgattttctgtaaaacaggagcaaagagaaattgtgtagaaacatgaggaagaccttcatattcttcatcacagattagaactacaggctgatagttcactaaataaattcaaacaaacggcttgggtaagattctaagaactttgttcca

SEQ ID NO: 2 - DNA, 158 bp, Homo sapiens
atagtttcgattttctgtaaaacaggagcaaagagaaattgtgaagaaacatgaggaagaccttcatattcttcatcacagattagaactacaggctgatagttcactaaataaattcaaacaaacggcttgggtaagattctaagaactttgttcca

SEQ ID NO: 3 - DNA, 108 bp, Homo sapiens
gagcaaagagaaattgtgtagaaacatgaggaagaccttcatattcttcatcacagattagaactacaggctgatagttcactaaataaattcaaacaaacggcttgg

SEQ ID NO: 4 - DNA, 108 bp, Homo sapiens
gagcaaagagaaattgtgaagaaacatgaggaagaccttcatattcttcatcacagattagaactacaggctgatagttcactaaataaattcaaacaaacggcttgg

SEQ ID NO: 5 - RNA, 22 nt, Artificial Sequence
  AON QRX136.27
ccuguuuuacagaaaaucgaaa

SEQ ID NO: 6 - RNA, 22 nt, Artificial sequence
  AON QRX136.28
cuuugcuccuguuuuacagaaa

SEQ ID NO: 7 - RNA, 21 nt, Artificial sequence
  AON QRX136.29 and QRX136.29a
cucuuugcuccuguuuuacag

SEQ ID NO: 8 - RNA, 22 nt, Artificial sequence
  AON QRX136.30
cuacacaauuucucuuugcucc

SEQ ID NO: 9 - RNA, 21 nt, Artificial sequence
  AON QRX136.31
uucuacacaauuucucuuugc

SEQ ID NO: 10 - RNA, 21 nt, Artificial sequence
  AON QRX136.32
cauguuucuacacaauuucuc

SEQ ID NO: 11 - RNA, 21 nt, Artificial sequence
  AON QRX136.33
cuuccucauguuucuacacaa

SEQ ID NO: 12 - RNA, 22 nt, Artificial sequence
  AON QRX136.34
gaaggucuuccucauguuucua

SEQ ID NO: 13 - RNA, 21 nt, Artificial sequence
  AON QRX136.35
gaauaugaaggucuuccucau

SEQ ID NO: 14 - RNA, 21 nt, Artificial sequence
  AON QRX136.36
aagaauaugaaggucuuccuc

SEQ ID NO: 15 - RNA, 21 nt, Artificial sequence
  AON QRX136.37
ugaagaauaugaaggucuucc

SEQ ID NO: 16 - RNA, 21 nt, Artificial sequence
  AON QRX136.38
gugaugaagaauaugaagguc

SEQ ID NO: 17 - RNA, 22 nt, Artificial sequence
  AON QRX136.39
cuaaucugugaugaagaauaug

SEQ ID NO: 18 - RNA, 21 nt, Artificial sequence
  AON QRX136.40
aguucuaaucugugaugaaga

SEQ ID NO: 19 - RNA, 22 nt, Artificial sequence
  AON QRX136.41
ccuguaguucuaaucugugaug

SEQ ID NO: 20 - RNA, 21 nt, Artificial sequence
  AON QRX136.42
gccuguaguucuaaucuguga

SEQ ID NO: 21 - RNA, 22 nt, Artificial sequence
  AON QRX136.43
cuaucagccuguaguucuaauc

SEQ ID NO: 22 - RNA, 21 nt, Artificial sequence
  AON QRX136.44
agugaacuaucagccuguagu

SEQ ID NO: 23 - RNA, 22 nt, Artificial sequence
  AON QRX136.45
uuuauuuagugaacuaucagcc

SEQ ID NO: 24 - RNA, 21 nt, Artificial sequence
  AON QRX136.46
aauuuauuuagugaacuauca

SEQ ID NO: 25 - RNA, 22 nt, Artificial sequence
  AON QRX136.47
ccguuuguuugaauuuauuuag

SEQ ID NO: 26 - RNA, 22 nt, Artificial sequence
  AON QRX136.48 and QRX136.48a
ccaagccguuuguuugaauuua

SEQ ID NO: 27 - RNA, 22 nt, Artificial sequence
  AON QRX136.49 and QRX136.49a
acccaagccguuuguuugaauu

SEQ ID NO: 28 - RNA, 22 nt, Artificial sequence
  AON QRX136.50 and QRX136.50a
cuuacccaagccguuuguuuga

SEQ ID NO: 29 - RNA, 21 nt, Artificial sequence
  AON QRX136.51 and QRX136.51a
cuuagaaucuuacccaagccg

SEQ ID NO: 30 - RNA, 21 nt, Artificial sequence
  AON QRX136.52
uggaacaaaguucuuagaauc

SEQ ID NO: 31 - DNA, 21 bp, Artificial sequence
  ddPCR primer/probe hCEP290_e34_ctrl_Fw
cgggcaacttgcaaatcacta

SEQ ID NO: 32 - DNA, 25 bp, Artificial sequence
  ddPCR primer/probe hCEP290_e35_ctrl_Hex
aatctgctttaaggttagcagaaca

SEQ ID NO: 33 - DNA, 20 bp, Artificial sequence
  ddPCR primer/probe hCEP290_e35_ctrl_Rv
ggcaatcgaagcctcagttc

SEQ ID NO: 34 - DNA, 20 bp, Artificial sequence
  ddPCR primer/probe hCEP290_e35_skip_Fw_2
accattgcaaacatgcaagc

SEQ ID NO: 35 - DNA, 23 bp, Artificial sequence
  ddPCR primer/probe hCEP290_e35-37_skip_Fam
tgtttcattaaatcctctctggc

SEQ ID NO: 36 - DNA, 20 bp, Artificial sequence
  ddPCR primer/probe hCEP290_e37_skip_Rv_2
tgttccatctcagccagacg

SEQ ID NO: 37 - RNA, 22 nt, Artificial sequence
  AON QRX136.30a
cuucacaauuucucuuugcucc

SEQ ID NO: 38 - RNA, 21 nt, Artificial sequence
  AON QRX136.33a
cuuccucauguuucuucacaa

SEQ ID NO: 39 - RNA, 22 nt, Artificial sequence
  AON QRX136.34a
gaaggucuuccucauguuucuu

SEQ ID NO: 40 - RNA, 21 nt, Artificial sequence
  AON QRX136.53a
caauuucucuuugcuccuguu

SEQ ID NO: 41 - RNA, 21 nt, Artificial sequence
  AON QRX136.54a
ggucuuccucauguuucuuca

SEQ ID NO: 42 - DNA, 11 bp, Homo sapiens
ggagcaaagag

SEQ ID NO: 43 - RNA, 11 nt, Artificial sequence
  Complementary sequence opposite to Box 1
cucuuugcucc

SEQ ID NO: 44 - DNA, 16 bp, Homo sapiens
tagaaacatgaggaag

SEQ ID NO: 45 - RNA, 16 nt, Artificial sequence
  Complementary sequence opposite Box 2 mutant
cuuccucauguuucua

SEQ ID NO: 46 - DNA, 16 bp, Homo sapiens
aagaaacatgaggaag

SEQ ID NO: 47 - RNA, 16 nt, Artificial sequence
  Complementary sequence opposite Box 2 wild type
cuuccucauguuucuu

SEQ ID NO: 48 - DNA, 17 bp, Homo sapiens
tcaaacaaacggcttgg

SEQ ID NO: 49 - RNA, 17 nt, Artificial sequence
  Complementary sequence opposite Box 3
ccaagccguuuguuuga

SEQ ID NO: 50 - RNA, 24 nt, Artificial sequence
  Negative control AON
gucccaucauucagguccauggca

SEQ ID NO: 51 - RNA, 20 nt, Artificial sequence
  AON QRX136.55a
aaggucuuccucauguuucu

SEQ ID NO: 52 - RNA, 18 nt, Artificial sequence
  AON QRX136.56a
aggucuuccucauguuuc

SEQ ID NO: 53 - RNA, 20 nt, Artificial sequence
  AON QRX136.57a
ccaagccguuuguuugaauu

SEQ ID NO: 54 - RNA, 18 nt, Artificial sequence
  AON QRX136.58a
ccaagccguuuguuugaa

SEQ ID NO: 55 - RNA, 16 nt, Artificial sequence
  AON QRX136.59a
ccaagccguuuguuug

SEQ ID NO: 56 - RNA, 20 nt, Artificial sequence
  AON QRX136.60a
uuacccaagccguuuguuug

SEQ ID NO: 57 - RNA, 18 nt, Artificial sequence
  AON QRX136.61a
cuuacccaagccguuugu

SEQ ID NO: 58 - RNA, 16 nt, Artificial sequence
  AON QRX136.62a
uuacccaagccguuug

SEQ ID NO: 59 - RNA, 19 nt, Artificial sequence
  AON QRX136.63a
uagaaucuuacccaagccg

SEQ ID NO: 60 - RNA, 17 nt, Artificial sequence
  AON QRX136.64a
gaaucuuacccaagccg

SEQ ID NO: 61 - RNA, 15 nt, Artificial sequence
  AON QRX136.65a
aucuuacccaagccg

SEQ ID NO: 62 - RNA, 19 nt, Artificial sequence
  AON QRX136.66a
aggucuuccucauguuucu

SEQ ID NO: 63 - RNA, 18 nt, Artificial sequence
  AON QRX136.67a
ggucuuccucauguuucu

SEQ ID NO: 64 - RNA, 17 nt, Artificial sequence
  AON QRX136.68a
gucuuccucauguuucu

SEQ ID NO: 65 - RNA, 16 nt, Artificial sequence
  AON QRX136.69a
ucuuccucauguuucu

SEQ ID NO: 66 - RNA, 17 nt, Artificial sequence
  AON QRX136.70a
ggucuuccucauguuuc

SEQ ID NO: 67 - RNA, 16 nt, Artificial sequence
  AON QRX136.71a
gucuuccucauguuuc

SEQ ID NO: 68 - RNA, 17 nt, Artificial sequence
  AON QRX136.72a
aggucuuccucauguuu

SEQ ID NO: 69 - RNA, 16 nt, Artificial sequence
  AON QRX136.73a
aggucuuccucauguu

SEQ ID NO: 70 - RNA, 18 nt, Artificial sequence
  AON QRX136.74a
aaggucuuccucauguuu

SEQ ID NO: 71 - RNA, 17 nt, Artificial sequence
  AON QRX136.75a
aaggucuuccucauguu

SEQ ID NO: 72 - RNA, 16 nt, Artificial sequence
  AON QRX136.76a
aaggucuuccucaugu

SEQ ID NO: 73 - RNA, 16 nt, Artificial sequence
  AON QRX136.77a
ggucuuccucauguuu

SEQ ID NO: 74 - RNA, 17 nt, Artificial sequence
  AON QRX136.78a
ccaagccguuuguuuga

SEQ ID NO: 75 - RNA, 18 nt, Artificial sequence
  AON QRX136.79a
uuacccaagccguuuguu

SEQ ID NO: 76 - RNA, 17 nt, Artificial sequence
  AON QRX136.80a
uuacccaagccguuugu

SEQ ID NO: 77 - RNA, 18 nt, Artificial sequence
  AON QRX136.81a_49
aaucuuacccaagccguu

SEQ ID NO: 78 - RNA, 17 nt, Artificial sequence
  AON QRX136.81a_50
aaucuuacccaagccgu

SEQ ID NO: 79 - RNA, 18 nt, Artificial sequence
  AON QRX136.82a
agaaucuuacccaagccg

SEQ ID NO: 80 - RNA, 16 nt, Artificial sequence
  AON QRX136.83a
uuagaaucuuacccaa

SEQ ID NO: 81 - RNA, 17 nt, Artificial sequence
  AON QRX136.84a
uuagaaucuuacccaag

SEQ ID NO: 82 - RNA, 18 nt, Artificial sequence
  AON QRX136.85a
uuagaaucuuacccaagc

SEQ ID NO: 83 - RNA, 16 nt, Artificial sequence
  AON QRX136.86a
uagaaucuuacccaag

SEQ ID NO: 84 - RNA, 16 nt, Artificial sequence
  AON QRX136.87a
agaaucuuacccaagc

SEQ ID NO: 85 - RNA, 18 nt, Artificial sequence
  AON QRX136.88a
uagaaucuuacccaagcc

SEQ ID NO: 86 - RNA, 17 nt, Artificial sequence
  AON QRX136.89a
agaaucuuacccaagcc

SEQ ID NO: 87 - RNA, 16 nt, Artificial sequence
  AON QRX136.90a
gaaucuuacccaagcc

SEQ ID NO: 88 - RNA, 20 nt, Artificial sequence
  AON m35ESE and m35ESEa
caugaaggucuuccucaugc

SEQ ID NO: 89 - RNA, 20 nt, Artificial sequence
  AON h35ESE and h35ESEa
uaugaaggucuuccucaugu

SEQ ID NO: 90 - RNA, 24 nt, Artificial sequence
  AON m35D and m35Da
guucucagaaucuuaccugagcug

SEQ ID NO: 91 - RNA, 24 nt, Artificial sequence
  AON h35D and h35Da
guucuuagaaucuuacccaagccg

SEQ ID NO: 92 - RNA, 22 nt, Artificial sequence
  AON H36D and H36Da
uagaaucuuacccaagccguuu

SEQ ID NO: 93 - RNA, 20 nt, Artificial sequence
  AON QRX136.101a
aagccguuuguuugaauuua

SEQ ID NO: 94 - RNA, 19 nt, Artificial sequence
  AON QRX136.102a
aagccguuuguuugaauuu

SEQ ID NO: 95 - RNA, 18 nt, Artificial sequence
  AON QRX136.103a
aagccguuuguuugaauu

SEQ ID NO: 96 - RNA, 17 nt, Artificial sequence
  AON QRX136.104a
aagccguuuguuugaau

SEQ ID NO: 97 - RNA, 16 nt, Artificial sequence
  AON QRX136.105a
aagccguuuguuugaa

SEQ ID NO: 98 - RNA, 15 nt, Artificial sequence
  AON QRX136.106a
aagccguuuguuuga

SEQ ID NO: 99 - RNA, 20 nt, Artificial sequence
  AON QRX136.107a
caagccguuuguuugaauuu

SEQ ID NO: 100 - RNA, 19 nt, Artificial sequence
  AON QRX136.108a
caagccguuuguuugaauu

SEQ ID NO: 101 - RNA, 18 nt, Artificial sequence
  AON QRX136.109a
caagccguuuguuugaau

SEQ ID NO: 102 - RNA, 17 nt, Artificial sequence
  AON QRX136.110a
caagccguuuguuugaa

SEQ ID NO: 103 - RNA, 16 nt, Artificial sequence
  AON QRX136.111a
caagccguuuguuuga

SEQ ID NO: 104 - RNA, 15 nt, Artificial sequence
  AON QRX136.112a
caagccguuuguuug

SEQ ID NO: 105 - RNA, 19 nt, Artificial sequence
  AON QRX136.113a
ccaagccguuuguuugaau

SEQ ID NO: 106 - RNA, 15 nt, Artificial sequence
  AON QRX136.114a
ccaagccguuuguuu

SEQ ID NO: 107 - RNA, 20 nt, Artificial sequence
  AON QRX136.115a
cccaagccguuuguuugaau

SEQ ID NO: 108 - RNA, 19 nt, Artificial sequence
  AON QRX136.116a
cccaagccguuuguuugaa

SEQ ID NO: 109 - RNA, 18 nt, Artificial sequence
  AON QRX136.117a
cccaagccguuuguuuga

SEQ ID NO: 110 - RNA, 17 nt, Artificial sequence
  AON QRX136.118a
cccaagccguuuguuug

SEQ ID NO: 111 - RNA, 16 nt, Artificial sequence
  AON QRX136.119a
cccaagccguuuguuu

SEQ ID NO: 112 - RNA, 15 nt, Artificial sequence
  AON QRX136.120a
cccaagccguuuguu

SEQ ID NO: 113 - RNA, 20 nt, Artificial sequence
  AON QRX136.121a
acccaagccguuuguuugaa

SEQ ID NO: 114 - RNA, 19 nt, Artificial sequence
  AON QRX136.122a
acccaagccguuuguuuga

SEQ ID NO: 115 - RNA, 18 nt, Artificial sequence
  AON QRX136.123a
acccaagccguuuguuug

SEQ ID NO: 116 - RNA, 17 nt, Artificial sequence
  AON QRX136.124a
acccaagccguuuguuu

SEQ ID NO: 117 - RNA, 16 nt, Artificial sequence
  AON QRX136.125a
acccaagccguuuguu

SEQ ID NO: 118 - RNA, 15 nt, Artificial sequence
  AON QRX136.126a
acccaagccguuugu

SEQ ID NO: 119 - RNA, 20 nt, Artificial sequence
  AON QRX136.127a
uacccaagccguuuguuuga

SEQ ID NO: 120 - RNA, 19 nt, Artificial sequence
  AON QRX136.128a
uacccaagccguuuguuug

SEQ ID NO: 121 - RNA, 18 nt, Artificial sequence
  AON QRX136.129a
uacccaagccguuuguuu

SEQ ID NO: 122 - RNA, 17 nt, Artificial sequence
  AON QRX136.130a
uacccaagccguuuguu

SEQ ID NO: 123 - RNA, 16 nt, Artificial sequence
  AON QRX136.131a
uacccaagccguuugu

SEQ ID NO: 124 - RNA, 15 nt, Artificial sequence
  AON QRX136.132a
uacccaagccguuug

SEQ ID NO: 125 - RNA, 19 nt, Artificial sequence
  AON QRX136.133a
uuacccaagccguuuguuu

SEQ ID NO: 126 - RNA, 15 nt, Artificial sequence
  AON QRX136.134a
uuacccaagccguuu

SEQ ID NO: 127 - RNA, 20 nt, Artificial sequence
  AON QRX136.135a
cuuacccaagccguuuguuu

SEQ ID NO: 128 - RNA, 19 nt, Artificial sequence
  AON QRX136.136a
cuuacccaagccguuuguu

SEQ ID NO: 129 - RNA, 17 nt, Artificial sequence
  AON QRX136.137a
cuuacccaagccguuug

SEQ ID NO: 130 - RNA, 16 nt, Artificial sequence
  AON QRX136.138a
cuuacccaagccguuu

SEQ ID NO: 131 - RNA, 15 nt, Artificial sequence
  AON QRX136.139a
cuuacccaagccguu

SEQ ID NO: 132 - RNA, 20 nt, Artificial sequence
  AON QRX136.140a
ucuuacccaagccguuuguu

SEQ ID NO: 133 - RNA, 19 nt, Artificial sequence
  AON QRX136.141a
ucuuacccaagccguuugu

SEQ ID NO: 134 - RNA, 18 nt, Artificial sequence
  AON QRX136.142a
ucuuacccaagccguuug

SEQ ID NO: 135 - RNA, 17 nt, Artificial sequence
  AON QRX136.143a
ucuuacccaagccguuu

SEQ ID NO: 136 - RNA, 16 nt, Artificial sequence
  AON QRX136.144a
ucuuacccaagccguu

SEQ ID NO: 137 - RNA, 15 nt, Artificial sequence
  AON QRX136.145a
ucuuacccaagccgu

SEQ ID NO: 138 - RNA, 20 nt, Artificial sequence
  AON QRX136.146a
aucuuacccaagccguuugu

SEQ ID NO: 139 - RNA, 19 nt, Artificial sequence
  AON QRX136.147a
aucuuacccaagccguuug

SEQ ID NO: 140 - RNA, 18 nt, Artificial sequence
  AON QRX136.148a
aucuuacccaagccguuu

SEQ ID NO: 141 - RNA, 17 nt, Artificial sequence
  AON QRX136.149a
aucuuacccaagccguu

SEQ ID NO: 142 - RNA, 16 nt, Artificial sequence
  AON QRX136.150a
aucuuacccaagccgu

SEQ ID NO: 143 - RNA, 20 nt, Artificial sequence
  AON QRX136.151a
aaucuuacccaagccguuug

SEQ ID NO: 144 - RNA, 19 nt, Artificial sequence
  AON QRX136.152a
aaucuuacccaagccguuu

SEQ ID NO: 145 - RNA, 16 nt, Artificial sequence
  AON QRX136.153a
aaucuuacccaagccg

SEQ ID NO: 146 - RNA, 15 nt, Artificial sequence
  AON QRX136.154a
aaucuuacccaagcc

SEQ ID NO: 147 - DNA, 30 bp, Homo sapiens
taaattcaaacaaacggcttgggtaagatt

SEQ ID NO: 148 - RNA, 20 nt, Artificial sequence
  Control AON
cuuaaagaugaucucuuacc

SEQ ID NO: 149 - RNA, 18 nt, Artificial sequence
  Control AON
agauucgcugcucuuguu